(19) United States
(12) Patent Application Publication
(10) Pub. No.: US 2025/0340648 A1
(43) Pub. Date: Nov. 6, 2025

(54) COMBINATION THERAPY FOR TREATMENT OF CANCER COMPRISING ANTI-PD-L1 AND ANTI-CD73 ANTIBODIES

(71) Applicant: MedImmune Limited, Cambridge (GB)

(72) Inventors: James EYLES, Cambridge (GB); Brajesh KAISTHA, Cambridge (GB); Simon DOVEDI, Cambridge (GB)

(21) Appl. No.: 19/126,249

(22) PCT Filed: Dec. 1, 2023
(86) PCT No.: PCT/IB2023/062127

§ 371 Date: Apr. 30, 2025

Related U.S. Application Data
(60) Provisional application No. 63/385,760, filed on Dec. 1, 2022.

(51) Int. Cl. C07K 16/28; A61K 31/337; A61K 31/513; A61K 31/555; A61K 39/00; A61P 35/00; G01N 33/68

(52) U.S. Cl. CPC C07K 16/2827; A61K 31/337; A61K 31/513; A61K 31/555; A61P 35/00; C07K 16/2896; G01N 33/6872; A61K 2039/507; C07K 2317/565; G01N 2333/70596

(57) ABSTRACT

The disclosure provides a method of treating a tumor in a subject comprising administering to the subject a therapeutically effective amount of a T-cell checkpoint inhibitor in combination with chemotherapy and/or radiotherapy; wherein the subject has decreased CD73 protein or CD73 activity levels compared to a normal subject. In some aspects, the method comprises further administering a CD73 inhibitor prior to, or concurrently with a combination of the T-cell checkpoint inhibitor and chemotherapy and/or radiotherapy.

### FIELD OF THE INVENTION

The invention disclosed herein relates to methods of treating a tumor in a subject comprising administration of a CD73 inhibitor, a T cell checkpoint inhibitor, and chemotherapy and/or radiotherapy.

### BACKGROUND OF THE INVENTION

Extracellular adenosine has emerged as an important regulator of immunological processes within the tumor microenvironment and is thought to diminish the effectiveness of T-cell checkpoint inhibiting drugs (Sidders B, et al., Clin Cancer Res 2020, 26:2176-87; Augustin R C, et al., J Immunother Cancer 2022, 10:e004089). Adenosine triphosphate (ATP), released from necrotic or damaged cells, is hydrolysed to adenosine by the sequential action of two ectonucleosidases working in tandem: CD39 (ENTPD1) and CD73 (NT5E). The resultant adenosine acts as a readily diffusible immunosuppressive ‘smog’, and it is likely that cytotoxic agents and radiotherapy exacerbate this process. To this point, enhanced anti-tumor activity is observed in preclinical models when radiotherapy is combined with an anti-CD73 (aCD73) inhibiting antibody treatment (Wennerberg E, et al. Cancer Immunol Res 2020; 8:465-78; and Wennerberg E, et al., Front Immunol 2017; 8:229.). Those studies highlighted an important role for radiation induced type-1 interferons in driving elevated tumoral cDC1 infiltration levels and a beneficial effect of CD73 inhibition on this biomarker when radiation activated type-1 interferon levels were suboptimal. Despite new information on the role of CD73 in the context of radiation-based standard-of-care, relatively little is known about the effects of adenosine pathway inhibiting drugs within the context of chemotherapy treatment regimens, even though a growing body of evidence points to an immunomodulatory axis for these agents (Coffelt S B, et al., Trends Immunol 2015; 36:198-216.). Inclusion of T-cell checkpoint inhibitors within this paradigm provides further scope for enhanced cell-mediated activity; by counteracting adaptive immune-resistance and unfettering anti-tumor immunity.

Oleclumab, a CD73 inhibiting human monoclonal IgG1-TM antibody (Hay C M, et al., Oncoimmunology 2016; 5) is currently in phase 2/3 of clinical development in combination with Durvalumab for treatment of patients with various solid tumors. Data from a Phase II platform study of Durvalumab in combination with Oleclumab in patients with unresectable stage III non-small-cell lung cancer, who had not progressed after prior chemoradiotherapy, highlighted significant benefit of the Oleclumab component. A phase 3 clinical trial is now underway in the same patient population. Therefore, there remains a need for effective therapy

### SUMMARY OF THE INVENTION

The present disclosure is directed to a method of inhibiting tumor growth in a subject in need thereof comprising administering to the subject a therapeutically effective amount of a PD-L1 inhibitor in combination with chemotherapy and/or radiotherapy; wherein the subject has decreased CD73 protein or CD73 activity levels compared to a normal subject.

The present disclosure is also directed to a method of treating cancer in a subject in need thereof comprising administering to the subject a therapeutically effective amount of a PD-L1 inhibitor in combination with chemotherapy and/or radiotherapy; wherein the subject has decreased CD73 protein or CD73 activity levels compared to a normal subject.

The present disclosure is also directed to a method of producing a protective tumor memory response in a subject comprising administering to the subject a therapeutically effective amount of a PD-L1 inhibitor in combination with chemotherapy and/or radiotherapy; wherein the subject has decreased CD73 protein or CD73 activity levels compared to a normal subject.

The present disclosure is also directed to a method of inhibiting tumor growth in a subject in need thereof comprising administering to the subject a therapeutically effective amount of a CD73 inhibitor, a PD-L1 inhibitor, and chemotherapy and/or radiotherapy.

The present disclosure is also directed to a method of treating cancer in a subject in need thereof comprising administering to the subject a therapeutically effective amount of a CD73 inhibitor, a PD-L1 inhibitor, and chemotherapy and/or radiotherapy.

The present disclosure is also directed to a method of producing a protective tumor memory response in a subject comprising administering to the subject a therapeutically effective amount of a CD73 inhibitor, a PD-L1 inhibitor, and chemotherapy and/or radiotherapy.

In one aspect, the PD-L1 inhibitor, and the chemotherapy and/or radiotherapy are administered concurrently. In another aspect, the PD-L1 inhibitor, and the chemotherapy and/or radiotherapy are administered sequentially. In another aspect, the CD73 inhibitor is administered prior to administration of the PD-L1 inhibitor, and the chemotherapy and/or radiotherapy. In one aspect, the chemotherapy is docetaxel, 5-fluorouracil, and/or oxaliplatin.

In one aspect, the PD-L1 inhibitor is an anti-PD-L1 antibody or an antigen-binding fragment thereof. In another aspect, the anti-PD-L1 antibody or antigen-binding fragment thereof comprising: (a) a heavy chain (HC) CDR1 comprising the amino acid sequence of SEQ ID NO:1, a HC CDR2 comprising the amino acid sequence of SEQ ID NO:2, and a HC CDR3 comprising the amino acid sequence of SEQ ID NO:3; and a light chain (LC) CDR1 comprising the amino acid sequence of SEQ ID NO:4, a LC CDR2 comprising the amino acid sequence of SEQ ID NO:5, and a LC CDR3 comprising the amino acid sequence of SEQ ID NO:6. In another aspect, the anti-PD-L1 antibody or antigen binding fragment thereof comprises a HC variable domain (VH) comprising the amino acid sequence of SEQ ID NO:7, and a LC variable domain (VL) comprising the amino acid sequence of SEQ ID NO:8. In another aspect, the anti-PD-L1 antibody is durvalumab.

In one aspect, the CD73 inhibitor is an anti-CD73 antibody or antigen-binding fragment thereof. In another aspect, the anti-CD73 antibody or antigen-binding fragment thereof comprising: (a) a HC CDR1 comprising the amino acid sequence of SEQ ID NO:9, a HC CDR2 comprising the amino acid sequence of SEQ ID NO:10, and a HC CDR3 comprising the amino acid sequence of SEQ ID NO:11; and a LC CDR1 comprising the amino acid sequence of SEQ ID NO: 12, a LC CDR2 comprising the amino acid sequence of SEQ ID NO:13, and a LC CDR3 comprising the amino acid sequence of SEQ ID NO: 14. In another aspect, the anti-CD73 antibody or antigen binding fragment thereof comprises a HC variable domain (VH) comprising the amino acid sequence of SEQ ID NO:15, and a LC variable domain (VL) comprising the amino acid sequence of SEQ ID NO: 16. In another aspect, the anti-CD73 antibody or antigen binding fragment thereof comprises a HC comprising the amino acid sequence of SEQ ID NO:17, and a LC comprising the amino acid sequence of SEQ ID NO: 18. In another aspect, the anti-CD73 antibody is oleclumab.

In one aspect, the administration results in upregulation of CXCR3 in the tumor microenvironment.

In one aspect, CD73 protein or CD73 activity levels are determined by immunohistochemistry (IHC), imaging mass cytometry (IMC), or mass spectroscopy imaging (MSI).

In another aspect, the tumor or cancer is a solid tumor or a cancer resulting from a solid tumor growth. In another aspect, the solid tumor is a lung tumor, breast tumor, colon tumor, bladder tumor, prostate tumor, colorectal tumor, head and neck tumor, liver tumor, or a pancreatic tumor. In another aspect, the lung tumor is a non-small cell lung tumor.

In one aspect, the subject is a human.

The present disclosure is also directed to the use of a CD73 inhibitor, a PD-L1 inhibitor, and chemotherapy and/or radiotherapy described herein for treating cancer in a subject in need thereof.

### BRIEF DESCRIPTION OF THE FIGURES

FIGS. 1A-1C show combined anti-CD73, anti-PD-L1 and 5FU+OHP treatment leads to enhanced complete responses in syngeneic mouse models. (A) Schematic of the experimental design. (B) BALB/c (CT26 cells in PBS) and (C) C57BL/6J mice (MCA205 cells in 50% Matrigel+PBS) were implanted with 5×10⁵ cells in the right flank and treated as shown in schematic. Growth curves are plotted from calliper measurements done thrice weekly. Addition of aCD73 and aPD-L1 to 5FU+OHP lead to significant increase in the number of complete responders (CR) in each model system-50% in CT26 (p=0.005 vs 5FU+OHP treated group) and 61.5% in MCA205 (p=0.008 vs 5FU+OHP treated group); Kruskal Wallis test.

FIGS. 2A-2B show responses in syngeneic mouse models to aCD73 and aPD-L1 treatments alone or in combination. BALB/c (CT26 cells in PBS (FIG. 2A)) and C57BL/6J mice (MCA205 cells in 50% Matrigel+PBS (FIG. 2B) were implanted with 500,000 cells in the right flank and treated as shown in schematic in FIG. 1A. Growth curves were plotted from calliper measurements done three times per week. Anti-CD73 monotherapy did not show any effect compared to control treated mice in both CT26 and MCA205 models. Anti-PD-L1 monotherapy had extremely modest response in CT26 (1/13 CR) only. Combined treatment with aCD73 and aPD-L1 also did not reveal any enhance response rate, with 1/13 CR mice observed in each CT26 and MCA205 tumor models.

FIGS. 3A-3B show that Mass Spectrometry Imaging (MSI) confirms adenosine pathway modulation by addition of anti-CD73 to 5FU+OHP. (3A) Schematic of the adenosine generation pathway. (3B) MSI images showing the abundance of ATP as well different metabolites of the adenosine pathway in the CT26 tumors. 5FU+OHP lead to modest increase in ATP and AMP abundance compared to control treated tumors however addition of aCD73 to 5FU+OHP resulted in much decreased adenosine as well inosine and xanthine.

FIGS. 4A-4E show that the addition of anti-CD73 to 5FU+OHP and Docetaxel does not lead to enhanced cytotoxicity in vitro. 10,000 cells of each HT-29 (4A), HCT-116 (4B), CT26 (4C and 4E) and MCA-205 (4D) cells were seeded in 96 well plate and treated with serially diluted chemotherapeutics as indicated along with anti-CD73. Cytotoxicity was measured by CellTiter-Glo® Luminescent assay after 72 hours of incubation with drugs. As shown in different panels of results, none of the cell lines tested showed any additivity of anti-CD73 to either 5FU+OHP and Docetaxel.

FIGS. 5A-5C show CD8 depletion leads to loss of efficacy seen in combined aCD73, aPD-L1 and 5FU+OHP treatment in syngeneic mouse model MCA205. (5A) Schematic of the experimental design. (5B) Growth curves in C57BL/6J mice of MCA205 tumor model (in 50% Matrigel+PBS 5×10⁵ cells) were plotted from calliper measurements done thrice weekly. Selective depletion of CD8 T cells resulted in decreased efficacy to the combination treatment leading to significantly shorter survival span (Kaplan Meier Plot, Log Rank test, p=0.02) compared to those where no CD8 cell depletion was carried out as shown in (5C).

FIGS. 6A-6D show IHC and MSI analysis confirms target (CD73) engagement and adenosine pathway modulation by addition of aCD73 to 5FU+OHP. (6A) Schematic of the experimental design. (6B) Immunohistochemistry analysis revealed lower levels of surface bound CD73 protein in the aCD73 to 5FU+OHP combination group. Results are expressed as area ratio of specific stain to background stain (hematoxylin). (6C) Mass Spectrometry Imaging showed a notable trend in adenosine, inosine and xanthine suppression as early PD biomarkers in the CT26 tumors from mice treated with anti-CD73 containing triple combination group. Results are reported as relative abundance in arbitrary units. (6D) Imaging mass cytometry highlighted that CT26 tumors from aCD73+5FU+OHP combination treated mice presented lower frequencies of cells expressing macrophage markers like CD68, F4/80 as well as suppressive tumor associated macrophage markers like CD163 and CD206 [left panel] as well as markers known to associate with cancer associated fibroblasts like collagen-iv, alpha smooth muscle actin, vimentin along with CD31. Results are expressed as mean+SEM of % positive cells.

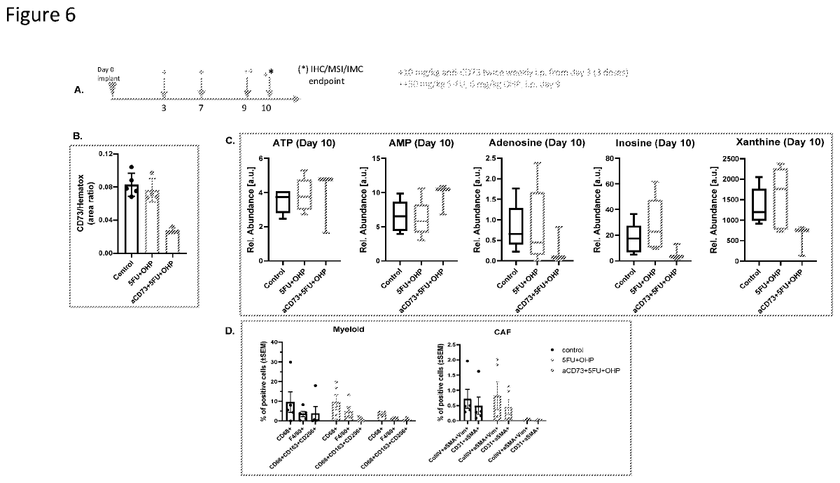

FIG. 7A shows the pharmacodynamic effect of aCD73+aPD-L1+5FU+OHP with respect to CT26 transcriptome. RNAseq analysis was used to see the changes in the CT tumor transcriptome. Upper panel shows the schematic of the experimental design. Lower panel shows contribution of the individual components in combined aCD73, aPD-L1 and 5FU+OHP treatment vs the aCD73+aPD-L1 group. As seen in left bottom panel, addition of aCD73 had the most profound effect leading to 589 differentially expressed (DE) genes as opposed to aPD-L1 which lead to only 35 DE genes (bottom right panel). Addition of the chemotherapeutic component to antibody doublet (aCD73+aPD-L1) lead to 546 DE genes. DE cut-off values used were Abs (log2FC)>=1 and Adj-pval<0.05.

FIG. 7B shows the transcriptome based deconvolution of key effects mediated by combination. Deconvolution of RNAseq analysis revealed the key pathways affected by addition of aCD73 and 5FU+OHP were the immune response activation pathways. Enriched pathways, KEGG (Kyoto Encyclopedia of Genes and Genomes) pathway enrichment KEGG, green) and Gene Ontology>biological process (GO BP, red), for the up-regulated genes are shown (DE cut-off values used were log2FC>=1 and Adj-pval<0.05).

FIGS. 7C shows the addition of aCD73 to aPD-L1 +5FU+OHP drove elevated tumor infiltrating lymphocytes (cytotoxic T-cell, NK, B-cells) and myeloid dendritic cells in CT26 tumors. Using the MCP counter tool the abundance of the different cell populations was estimated. As shown in the heatmap, addition of aCD73 had the most profound effect on driving the relevant immune cells like-cytotoxic T-cells, NK-cells, B-cells and myeloid dendritic cells (DC). The DC infiltration effect was solely mediated by addition of the aCD73 to the 5FU+OHP, as addition of aPD-L1 to 5FU+OHP didn't result in increased DC infiltration in the tumors.

FIGS. 8A-8B show that combined aCD73, aPD-L1 and Docetaxel (DTX) treatment leads to enhanced complete responses in CT26 syngeneic tumor model. (8A) Schematic of the experimental design. (8B) BALB/c were implanted with 5×10⁵ CT26 cells in PBS and treated shown in schematic. Growth curves were plotted from calliper measurements done thrice weekly. Addition of aCD73 and aPD-L1 to docetaxel lead to significant (p=0.0001 vs vehicle) increase in the number of complete responders (CR) 58% compared to 25% CR seen upon addition of aPD-L1 alone to docetaxel (p=0.001); Kruskal Wallis test.

FIGS. 9A-9B show that concurrent treatment with aCD73, aPD-L1 and radiotherapy (RTx) leads to enhanced complete responses in MC38 syngeneic tumor model. (9A) Schematic of the experimental design. (9B) C57BL6/J were implanted with 5×10⁵ MC38 cells in PBS and treated shown in schematic. Growth curves were plotted from calliper measurements done thrice weekly. Concurrent treatment with aCD73, aPD-L1 and radiotherapy lead to significant (p-0.0001 vs NT) number of complete responders (CR) 58% compared to none see in RTx alone group (p=0.5 vs NT), Kruskal Wallis test.

FIG. 10 shows imaging mass cytometry (IMC) of the pharmacodynamic changes observed by addition of aCD73 to 5FU+OHP. IMC images of the CT26 tumors bearing mice treated with either control, 5FU+OHP and aCD73+5FU+OHP. aCD73+5FU+OHP combination treated mice tumors presented lower frequencies of cells expressing macrophage markers like CD68, F4/80 as well as suppressive tumor associated macrophage markers like CD163 and CD206 [left panel] as well as markers known to associate with cancer associated fibroblasts like collage-iv, alpha smooth muscle actin, vimentin along with CD31.

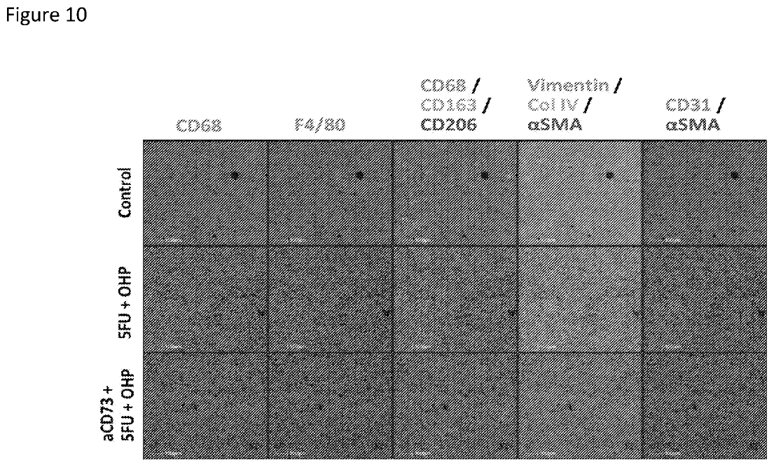

FIG. 11 shows the top 50 differentially expressed genes for Triple vs Control were identified and log₂ fold changes shown for different comparisons. Heatmap on the right panel shows expression change for selected immune-related genes across different conditions.

FIG. 12 shows a schematic of treatment groups divided by timing of aCD73, aPD-L1 and radiotherapy determined using a MC38 syngeneic mouse model. Six groups of mice were implanted with 5×10⁵ cells as shown.

FIG. 13 shows that mice concurrently treated with aCD73, aPD-L1, and RTx showed the highest level of tumor inhibition and subsequent probability of survival.

FIG. 14 shows that mice concurrently treated with aCD73, aPD-L1, and RTx also demonstrated an induction of a protective memory response upon rechallenge using B16F10 and MC38 mouse models.

FIG. 15 shows a schematic of treatment groups divided by timing of the individual aCD73, aPD-L1, and radiotherapy. As before, MC38 cells were implanted as before and the timing of the individual therapies was mapped.

FIG. 16 shows administration of aCD73 therapy prior to aPD-L1 and RTx results in the greatest decrease in tumor volume, which correlated to the highest probability of survival.

### DETAILED DESCRIPTION OF THE INVENTION

Using mouse models of cancer and a murine surrogate of Oleclumab (hereafter called aCD73), the effects of CD73 inhibition were explored in combination with chemotherapies, or radiotherapy, and PD-L1 blockade. As a corollary, the same approach was explored to determine if it can be applied to enhance the effects of radiotherapy. As described herein, these combinations were highly effective in terms of improved tumor growth inhibition, induction of a protective memory response, and overall survival benefit. Transcriptomic based pharmacodynamic assessments highlighted increased abundances of cytotoxic lymphocytes and immune-supportive myeloid populations in the tumor. Profiling of treatment groups representing the various components of the combination allowed deconvolution of contributing individual therapeutic components; highlighting effects conferred by CD73 inhibition in the context of the combined chemotherapy and PD-L1 blockade.

### 1. Definitions

In order that the present disclosure may be more readily understood, certain terms are first defined. As used in this application, except as otherwise expressly provided herein, each of the following terms shall have the meaning set forth below. Additional definitions are set forth throughout the application.

Before describing the present disclosure in detail, it is to be understood that this disclosure is not limited to specific compositions or process steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety.

It is understood that wherever aspects are described herein with the language “comprising,” otherwise analogous aspects described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. A heavy chain may have the C-terminal lysine or not. Unless specified otherwise herein, the amino acids in the variable regions are numbered using the Kabat numbering system and those in the constant regions are numbered using the EU system.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” includes monospecific, bispecific, or multi-specific antibodies, as well as a single chain antibody. In aspects, the antibody is a bispecific antibody. In other aspects, the antibody is a monospecific antibody.

As used herein, an “IgG antibody” has the structure of a naturally occurring IgG antibody, i.e., it has the same number of heavy and light chains and disulfide bonds as a naturally occurring IgG antibody of the same subclass. For example, an anti-ICOS IgG1, IgG2, IgG3 or IgG4 antibody consists of two heavy chains (HCs) and two light chains (LCs), wherein the two heavy chains and light chains are linked by the same number and location of disulfide bridges that occur in naturally occurring IgG1, IgG2, IgG3 and IgG4 antibodies, respectively (unless the antibody has been mutated to modify the disulfide bonds)

An “isolated antibody” refers to an antibody that is substantially free of other antibodies having different antigenic specificities (e.g., an isolated antibody that binds specifically to PD-L1 is substantially free of antibodies that bind specifically to antigens other than PD-1). An isolated antibody that binds specifically to PD-L1 may, however, have cross-reactivity to other antigens, such as PD-L1 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-PD-LI antibody binds specifically to PD-L1 and an anti-CD73 antibody binds specifically to CD73.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-CD73 antibody described herein, include:
(1) a Fab fragment (fragment from papain cleavage) or a similar monovalent fragment consisting of the VL, VH, LC and CHI domains;
(2) a F (ab') 2 fragment (fragment from pepsin cleavage) or a similar bivalent fragment comprising two Fab fragments linked by a disulfide bridge at the hinge region;
(3) a Fd fragment consisting of the VH and CHI domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “CD73 polypeptide” as used herein refers to the CD73 (Cluster of Differentiation 73) protein, also referred to as 5 ‘-nucleotidase (5’-NT) or ecto-5′-nucleotidase in the literature, which is encoded by the NT5E gene. See, e.g., Misumi et al. Eur. J. Biochem. 191(3): 563-9 (1990). The respective sequences of the human and murine forms of CD73 are available at the Uniprot database under accession numbers P21589 and Q61503, respectively. In defining any CD73 antibody epitopes, the amino acid numbering used represents the amino acid residue of the mature CD73 protein which does not contain the signal sequence residues. Accordingly, an antibody binding amino acids Val144, Lys180, and Asn185, for example, refers to the amino acid positions after cleavage of the signal sequence, i.e., the amino acid in the mature protein.

A “T-cell checkpoint inhibitor” or “immune checkpoint inhibitor” refers to any compound inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and 5 analogs having at least one common epitope with hPD-L1. The complete hPD-L1 sequence can be found under GenBank Accession No. Q9NZQ7.

A “patient” as used herein includes any patient who is afflicted with a cancer (e.g., non-small cell lung cancer (NSCLC)). The terms “subject” and “patient” are used interchangeably herein.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment may refer to alleviation of at least one symptom of a solid tumor. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-CD73 antibody and the amount of anti-PD-L1 antibody, in combination, clinically proven to affect a significant decrease in cancer or slowing of progression of cancer, such as an advanced solid tumor. The term “progression-free survival,” which can be abbreviated as PFS, as used herein refers to the length of time during and after the treatment of a solid tumor (i.e., NSCLC) that a patient lives with the disease but it does not get worse.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “tumor” as used herein refers to any mass of tissue that results from excessive cell growth or proliferation, either benign (non-cancerous) or malignant (cancerous), including pre-cancerous lesions.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

Various aspects of the disclosure are described in further detail in the following subsections.

### 2. Methods of the Disclosure

In one aspect, the present disclosure is directed to a method for inhibiting tumor growth in a subject having decreased CD73 protein expression or CD73 activity levels compared to a normal subject. A combination therapy of a T-cell checkpoint inhibitor and chemotherapy and/or radiotherapy results in better therapeutic outcomes (e.g., objective response rate and disease control rate). In order to improve the treatment of malignant tumors, in one aspect, the present disclosure provides identifying a patient as having decreased CD73 protein expression or CD73 activity and providing a combination therapy of a T-cell checkpoint inhibitor and chemotherapy and/or radiotherapy.

A wide variety of chemotherapeutic agents may be used in accordance with the present aspects. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

In another aspect, the combination includes radiotherapy. Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In one aspect, the present disclosure is directed to identifying a patient as having decreased CD73 protein expression or CD73 activity levels and treating the subject by administering a T-cell checkpoint inhibitor (e.g., an anti-PD-L1 antibody) and chemotherapy and/or radiotherapy. In one aspect, the disclosure includes a method of identifying a patient as having decreased CD73 protein expression or CD73 activity levels and treating the subject by administering an anti-PD-L1 antibody and chemotherapy and/or radiotherapy.

In another aspect, the present disclosure is directed to a method for inhibiting tumor growth in a subject comprising administering a combination therapy of a CD73 inhibitor, a T-cell checkpoint inhibitor and chemotherapy and/or radiotherapy. In one aspect, tumor growth in the subject is inhibited by administering an anti-CD73 antibody, an anti-PD-L1 antibody, and chemotheraphy and/or radiotherapy.

CD73 is a glycosylphosphatidylinositol (GPI) anchored cell surface protein that catalyzes the hydrolysis of adenosine monophosphate (AMP) to adenosine, and works in concert with CD39, which converts adenosine triphosphate (ATP) into AMP. The resulting adenosine functions as a signaling molecule that activates the P1 receptors expressed on the cell surface in many different tissues. Four G protein-coupled P1 or adenosine receptors have been cloned and designated as A1, A2A, A2B, and A3. Adenosine impacts a wide range of physiological processes including neural function, vascular perfusion, and immune responses. In doing so, this metabolite regulates CNS, cardiovascular, and immune system functions, to name a few.

Increasing evidence suggests that interactions between tumor cells and their microenvironment are essential for tumorigenesis. The purinergic signaling pathway in which CD73 plays a critical role, has emerged as an important player in cancer progression. It has become clear in recent years that adenosine is one of the most important immunosuppressive regulatory molecules in the tumor microenvironment and contributes to immune escape and tumor progression.

CD73 is a key protein molecule in cancer development. CD73 has been found to be overexpressed in many cancer cell lines and tumor types including, for example, breast cancer, colorectal cancer, ovarian cancer, gastric cancer, gallbladder cancer, and cancers associated with poor prognosis.

In addition to being a prognostic biomarker in cancer patients, overexpression of CD73 has also been found to be functionally linked to therapy (e.g., cancer therapy) resistance. Elevated levels of CD73 were initially linked to resistance to a variety of chemotherapeutic agents including vincristine and doxorubicin.

CD73 has also been shown to be involved in immunotherapy resistance. This ectonucleotidase participates in the process of tumor immune escape by inhibiting the activation, clonal expansion, and homing of tumor-specific T cells (in particular, T helper and cytotoxic T cells); impairing tumor cell killing by cytolytic effector T lymphocytes; driving, via pericellular generation of adenosine, the suppressive capabilities of Treg and Th17 cells; enhancing the conversion of type 1 macrophages into tumor-promoting type 2 macrophages; and promoting the accumulation of MDSCs.

In some aspects of the disclosure, the subjects to be treated have decreased CD73 protein expression or CD73 activity. In some aspects, this decreased is caused by prior treatment with a CD73 inhibitor. In some aspects, the CD73 inhibitor is an anti-CD73 antibody or antigen-binding fragment thereof.

In some aspects, the anti-CD73 antibody or antigen-binding fragment thereof is an antibody described in PCT Publication No. WO 2016/075099. In some aspects, the anti-CD73 antibody comprises the HC CDR1-3 and LC CDR 1-3 of SEQ ID NOs: 9-11 and 12-14, respectively. In some aspects, the anti-CD73 antibody comprises the VH and VL of SEQ ID NOs: 15 and 16, respectively. In some aspects, the anti-CD73 antibody comprises the heavy and light chains of SEQ ID NOs: 17 and 18, respectively. In some aspects, the anti-CD73 antibody is oleclumab.

In some aspects, the method comprises administering an anti-CD73 antibody or antigen-binding fragment thereof, prior to, or concurrently with a T-cell checkpoint inhibitor and chemotherapy and/or radiotherapy. In some aspects, the anti-CD73 antibody or antigen-binding fragment thereof is an antibody described in PCT Publication No. WO 2016/075099. In some aspects, the anti-CD73 antibody comprises the HC CDR1-3 and LC CDR1-3 of SEQ ID NOs: 9-11 and 12-14, respectively. In some aspects, the anti-CD73 antibody comprises the VH and VL of SEQ ID NOs: 15 and 16, respectively. In some aspects, the anti-CD73 antibody comprises the heavy and light chains of SEQ ID NOs: 17 and 18, respectively. In some aspects, the anti-CD73 antibody is oleclumab.

In some aspects, prior art antibodies can be used to decrease CD73 expression and/or activity. Exemplary anti-CD73 antibodies are described in PCT Publication Nos. WO 2018/137598; WO 2016/081748; WO 2017/064043, WO 2017/100670, and WO 2018/237157.

In one aspect, the disclosure includes a method of selecting a tumor in a human patient for immunotherapy, comprising: (a) determining the level of CD73 protein expression or CD73 activity in a tumor sample; and (b) selecting the tumor for immunotherapy if the tumor sample displays decreased CD73 protein expression or CD73 activity. In one aspect, the disclosure includes a method of identifying a tumor in a human patient that is likely to be responsive to an immunotherapy, the method comprising: (a) determining the level of CD73 protein expression or CD73 activity in a tumor sample; and (b) identifying the tumor as likely to be responsive to treatment if the tumor displays decreased CD73 protein expression or CD73 activity. In some aspects, the immunotherapy comprises contacting the tumor with a therapeutically effective amount of a PD-1 pathway inhibitor. In some aspects, the immunotherapy comprises contacting the tumor with a therapeutically effective amount of an anti-PD-L1 antibody. In some aspects, the immunotherapy comprises contacting the tumor with a therapeutically effective amount of an anti-PD-1 antibody. In some aspects, the immunotherapy comprises contacting the tumor with a therapeutically effective amount of an anti-CTLA-4 antibody. In some aspects, the immunotherapy comprises contacting the tumor with a therapeutically effective amount of a PD-1 pathway inhibitor and chemotherapy and/or radiotherapy.

In another aspect, the disclosure includes a method for treating tumor growth in a human patient in need thereof comprising administering to the patient a T-cell checkpoint inhibitor and chemotherapy and/or radiotherapy, wherein the patient is identified as having decreased CD73 protein expression or CD73 activity prior to the administration. In some aspects, the T-cell checkpoint therapy comprises administering a therapeutically effective amount of a PD-1 pathway inhibitor. In some aspects, the T-cell checkpoint therapy comprises administering a therapeutically effective amount of an anti-PD-L1 antibody.

In still other aspects, the disclosure includes a method for reducing a tumor size at least by 10% in a human patient afflicted with a tumor comprising administering to the patient a combination therapy disclosed herein (e.g., an anti-CD73 antibody, an anti-PD-L1 antibody, and chemotherapy and/or radiotherapy; or an anti-PD-L1 antibody, and chemotherapy and/or radiotherapy). In some aspects, the patient has been identified as having decreased CD73 protein expression or CD73 activity prior to the administration and wherein the administration reduces the tumor size at least about 10%, about 20%, about 30%, about 40%, about 50%, about 60%, about 70%, about 80%, about 90%, or 100% compared to the tumor size prior to the administration.

The disclosure can also include a method of preventing a relapse and/or inducing remission in a patient comprising administering to the patient a combination therapy disclosed herein (e.g., an anti-CD73 antibody, an anti-PD-L1 antibody, and chemotherapy and/or radiotherapy; or an anti-PD-L1 antibody, and chemotherapy and/or radiotherapy). In some aspects, the method of the disclosure comprises (i) identifying a patient as having decreased CD73 protein expression or CD73 activity; (ii) administering to the patient a combination therapy disclosed herein (e.g., an anti-CD73 antibody, an anti-PD-L1 antibody, and chemotherapy and/or radiotherapy; or an anti-PD-L1 antibody, and chemotherapy and/or radiotherapy).

The methods of the disclosure, as a result of the administration of a combination therapy disclosed herein, can treat the malignant tumor, reduce the tumor size, prevent growth of the tumor, eliminate the tumor from the patient, prevent a relapse of a tumor, induce a remission in a patient, or any combination thereof. In certain aspects, the administration of a combination therapy disclosed herein induces a complete response. In other aspects, the administration of the combination therapy disclosed herein induces a partial response. In some aspects, the immunotherapy comprises administering a therapeutically effective amount of a PD-1 pathway inhibitor and chemotherapy and/or radiotherapy. In some aspects, the PD-1 pathway inhibitor is an anti-PD-L1 antibody. In some aspects, the combination therapy comprises administering a therapeutically effective amount of a CD73 inhibitor, a T-cell checkpoint inhibitor, and chemotherapy and/or radiotherapy. In some aspects, the combination therapy comprises administering a therapeutically effective amount of an anti-CD73 antibody, an anti-PD-L1 antibody, and chemotherapy and/or radiotherapy.

In some aspects, CD73 expression or CD73 activity is determined by receiving the results of an assay capable of determining CD73 expression/activity.

### Measurement of CD73 Activity/Expression

In order to assess the CD73 expression/activity, in one aspect, a test sample is obtained from the patient who is in need of the therapy. In some aspects, a test sample includes, but is not limited to, any clinically relevant sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test sample is from a metastasis. In some aspects, test samples are taken from a subject at multiple time points, for example, before treatment, during treatment, and/or after treatment. In some aspects, test samples are taken from different locations in the subject, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In another aspect, the assessment of CD73 activity/expression can be achieved without obtaining a test tissue sample. In some aspects, selecting a suitable patient includes (i) optionally providing a test tissue sample obtained from a patient with cancer of the tissue, the test tissue sample comprising tumor cells; and (ii) assessing the proportion of cells in the test tissue sample that express CD73 on the surface of the cells based on an assessment that the proportion of cells in the test tissue sample that express CD73 on the cell surface is lower than a predetermined threshold level.

In any of the methods comprising the measurement of CD73 expression in a test sample, however, it should be understood that the step comprising the provision of a test sample obtained from a patient is an optional step. That is, in certain aspects the method includes this step, and in other aspects, this step is not included in the method. It should also be understood that in certain aspects the “measuring” or “assessing” step to identify, or determine the number or proportion of, cells in the test sample that express CD73 is performed by a transformative method of assaying for CD73 expression, for example by performing a reverse transcriptase-polymerase chain reaction (RT-PCR) assay, IHC, imaging mass cytometry (IMC), or a mass spectroscopy imaging (MSI) assay. In certain other aspects, no transformative step is involved and CD73 expression is assessed by, for example, reviewing a report of test results from a laboratory. In some aspects, CD73 activity/expression is assessed by reviewing the results of, for example, an immunohistochemistry assay from a laboratory. In certain aspects, the steps that provide the test result is performed by a medical practitioner or someone acting under the direction of a medical practitioner. In other aspects, these steps are performed by an independent laboratory or by an independent person such as a laboratory technician.

In certain aspects of any of the present methods, the proportion of cells that express CD73 is assessed by performing an assay to detect the presence of CD73 RNA. In further aspects, the presence of CD73 RNA is detected by RT-PCR, in situ hybridization or RNase protection. In some aspects, the presence of CD73 RNA is detected by an RT-PCR based assay. In some aspects, scoring the RT-PCR based assay comprises assessing the level of CD73 RNA expression in the test tissue sample relative to a predetermined level.

In other aspects, the proportion of cells that express CD73 is assessed by performing an assay to detect the presence of CD73 polypeptide. In further aspects, the presence of CD73 polypeptide is detected by IHC, enzyme-linked immunosorbent assay (ELISA), in vivo imaging, or flow cytometry. In some aspects, CD73 expression is assayed by IHC, imaging mass cytometry (IMC), or mass spectroscopy imaging (MSI). In other aspects of all of these methods, cell surface expression of CD73 is assayed using, e.g., IHC or in vivo imaging.

### T-Cell Checkpoint Inhibitors

In the tumor microenvironment, cancer cells can evade the immunosurveillance by changing their surface antigens, thus avoiding the detection and destruction by host lymphocytes. A central mechanism of tumor-induced immune suppression is the increased expression of ligands able to bind inhibitory T cell receptors. These ligands are known as T-cell or immune checkpoints and act in physiological conditions to prevent the development of autoimmunity at multiple steps during the immunological response. The main mechanisms involved in the T cell modulation are the suppression of potential autoreactive naïve T cell (characterized by a TCR directed against self-antigens) at initial stages in lymph nodes, or in later phases the T cell deactivation in peripheral tissues. This process is called peripheral tolerance and is exerted mainly by the immune checkpoints cytotoxic T-lymphocyte-associated antigen 4 (CTLA-4) and programmed death 1 (PD-1) pathways. Tumor cells have developed ways to take advantage of peripheral tolerance by inducing a deranged immune checkpoint expression by T cell in order to avoid immune recognition.

Other novel checkpoints have also been discovered. The next generation of immune checkpoints includes, for example, the lymphocyte activation gene-3 (LAG-3), T cell immunoglobulin and mucin-domain containing-3 (TIM-3), B and T cell lymphocyte attenuator (BTLA), T cell immunoglobulin and ITIM domain (TIGIT), V-domain Ig suppressor of T cell activation (VISTA), and B7 homolog 3 protein (B7-H3).

### PD-1 Pathway Inhibitors

In certain aspects, the present application encompasses use of an anti-PD-L1 antibody as the T-cell checkpoint inhibitor. In one aspect, the anti-PD-L1 antibody inhibits the binding of PD-L1 receptor, i.e., PD-1 to its ligand PD-L1.

Anti-human-PD-L1 antibodies (or VH and/or VL domains derived therefrom) suitable for use in the methods of the disclosure can be generated using methods well known in the art. In certain aspects, the anti-PD-L1 antibody or antigen-binding fragment thereof is an antibody described in PCT Publication No. WO 2011/066389. In some aspects, the anti-PD-L1 antibody comprises the HC CDR1-3 and LC CDR1-3 of SEQ ID NOs: 1-3 and 4-6, respectively. In some aspects, the anti-PD-L1 antibody comprises the VH and VL of SEQ ID NOs: 7 and 8, respectively. In some aspects, the anti-CD73 antibody is durvalumab.

Alternatively, art recognized anti-PD-L1 antibodies can be used. For example, anti-PD-L1 antibodies useful in the claimed methods are disclosed in U.S. Pat. No. 7,943,743. Such anti-PD-L1 antibodies include 12A4 (also referred to as BMS-936559). In some aspects, the anti-PD-L1 antibody is atezolizumab (Tecentriq or RG7446) (see, e.g., Herbst et al. (2013) J Clin Oncol 31 (suppl): 3000. Abstract; U.S. Pat. No. 8,217,149), or avelumab (Bavencio). Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, which are herein incorporated by reference. Antibodies that compete with any of these art-recognized antibodies or inhibitors for binding to PD-L1 also can be used.

In certain aspects, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

In certain aspects, the PD-L1 antibody is durvalumab (IMFINZI™). Durvalumab is a human IgGI kappa monoclonal anti-PD-L1 antibody.

In certain aspects, the PD-L1 antibody is atezolizumab (TECENTRIQ®). Atezolizumab is a fully humanized IgGI monoclonal anti-PD-L1 antibody.

In certain aspects, the PD-L1 antibody is avelumab (BAVENCIO®). Avelumab is a human IgGI lambda monoclonal anti-PD-L1 antibody.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., durvalumab, atezolizumab, and/or avelumab. In some aspects, the anti-PD-L1 antibody binds the same epitope as any of the anti-PD-L1 antibodies described herein, e.g., durvalumab, atezolizumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, durvalumab, atezolizumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with durvalumab, atezolizumab, and/or avelumab for binding to human PD-L1.

3. PD-1 Inhibitors

In some aspects, the T-cell check point inhibitor is a PD-1 pathway inhibitor, for example an anti-PD-1 antibody. In some aspects, the PD-1 pathway inhibitor is a PD-L2-binding agent, for example an anti-PD-L2 antibody. In further aspects, the PD-L1-binding agent is a soluble PD-1 polypeptide, for example, a PD-1-Fc fusion polypeptide capable of binding to PD-L1. In further aspects, the PD-L2-binding agent is a soluble PD-1 polypeptide, for example, a PD-1-Fc fusion polypeptide capable of binding to PD-L2.

Anti-human-PD-1 antibodies (or VH and/or VL domains derived therefrom) suitable for use in the disclosure can be generated using methods well known in the art. Alternatively, art recognized anti-PD-1 antibodies can be used.

In other aspects, the anti-PD-1 antibody is Nivolumab or BMS-936558 described in WO 2006/121168. Other known PD-1 antibodies include lambrolizumab (MK-3475) described in WO 2008/156712. Further known PD-1 antibodies and other PD-1 inhibitors include those described in, for example, WO 2009/014708, WO 03/099196, WO 2009/114335 and WO 2011/161699, which are herein incorporated by reference. In one aspect, the anti-PD-1 antibody is REGN2810. In one aspect, the anti-PD-1 antibody is PDR001. Another known anti-PD-1 antibody is pidilizumab (CT-011).

In some aspects, the anti-PD-1 antibody or fragment thereof cross-competes with pembrolizumab. In some aspects, the anti-PD-1 antibody or fragment thereof binds to the same epitope as pembrolizumab. In certain aspects, the anti-PD-1 antibody has the same CDRs as pembrolizumab. In another aspect, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587.

In certain aspects, the first antibody is an anti-PD-1 antagonist. One example of the anti-PD-1 antagonist is AMP-224, which is a B7-DC Fc fusion protein. AMP-224 is discussed in U.S. Publ. No. 2013/0017199.

In other aspects, the anti-PD-1 antibody or fragment thereof cross-competes with BGB-A317. In some aspects, the anti-PD-1 antibody or fragment thereof binds the same epitope as BGB-A317. In certain aspects, the anti-PD-1 antibody has the same CDRs as BGB-A317. In certain aspects, the anti-PD-1 antibody is BGB-A317, which is a humanized monoclonal antibody. BGB-A317 is described in U.S. Publ. No. 2015/0079109.

In some aspects, the antibody is pidilizumab (CT-011), which is an antibody previously reported to bind to PD-1 but which is believed to bind to a different target. pidilizumab is described in US Pat. No. 8,686,119 B2 or WO 2013/014668 A1.

In certain aspects, the antibodies that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Anti-PD-1 antibodies useful for the compositions of the disclosure also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody.

Anti-PD-1 antibodies usable in the disclosed methods also include isolated antibodies that bind specifically to human PD-1 and cross-compete for binding to human PD-1 with any anti-PD-1 antibody disclosed herein. In some aspects, the anti-PD-1 antibody binds the same epitope as any of the anti-PD-1 antibodies described herein. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody.

### 4. LAG-3 Inhibitors

In some aspects, a LAG-3 inhibitor is a LAG-3-binding agent, for example an anti-LAG-3 antibody. In some aspects, the LAG-3 inhibitor is a soluble LAG-3 polypeptide, for example, a LAG-3-Fc fusion polypeptide capable of binding to MHC Class II.

Anti-human-LAG-3 antibodies (or VH/VL domains derived therefrom) suitable for use in the disclosure can be generated using methods well known in the art. Alternatively, art recognized anti-LAG-3 antibodies can be used. In certain aspects, LAG-3 inhibitors include an anti-LAG-3 bispecific antibody.

In some aspects, the anti-LAG-3 antibody is relatlimab or BMS-986016 comprising heavy and light chains described in PCT/US13/48999.

In another aspect, the antibody competes for binding with and/or binds to the same epitope on LAG-3 as the above-mentioned antibodies.

In some aspects, art recognized anti-LAG-3 antibodies can be used in the therapeutic methods of the disclosure. For example, the anti-human LAG-3 antibody described in US2011/0150892 A1, and referred to as monoclonal antibody 25F7 (also known as “25F7” and “LAG-3.1) can be used. Other art recognized anti-LAG-3 antibodies that can be used include IMP731 (H5L7BW) described in US 2011/007023, MK-4280 (28G-10) described in WO2016028672, REGN3767 described in Journal for ImmunoTherapy of Cancer, (2016) Vol. 4, Supp. Supplement 1 Abstract Number: P195, BAP050 described in WO2017/019894, IMP-701 (LAG-525), Sym022, TSR-033, MGD013, BI754111, FS118, AVA-017 and GSK2831781. These and other anti-LAG-3 antibodies useful in the claimed disclosure can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, and WO2014/140180. In one aspect, the LAG-3 inhibitor is IMP321 (eftilagimod alpha). The contents of each of these references are incorporated by reference herein in their entirety.

Antibodies that compete with any of the above-referenced art-recognized antibodies for binding to LAG-3 also can be used.

### 5. CTLA-4 Antagonists

In certain aspects, the present application encompasses use of an anti-CTLA-4 antibody. In one aspect, the anti-CTLA-4 antibody binds to and inhibits CTLA-4. In some aspects, the anti-CTLA-4 antibody is ipilimumab (YERVOY), tremelimumab (ticilimumab; CP-675,206), AGEN-1884, or ATOR-1015.

In one aspect, the CTLA-4 antagonist is a soluble CTLA-4 polypeptide. In one aspect, the soluble CTLA-4 polypeptide is abatacept (Orencia), belatacept (Nulojix), RG2077, or RG-1046. In another aspect, the CTLA-4 antagonist is a cell based therapy. In some aspects, the CTLA-4 antagonist is an anti-CTLA-4 mAb RNA/GITRL RNA-transfected autologous dendritic cell vaccine or an anti-CTLA-4 mAb RNA-transfected autologous dendritic cell vaccine.

### 6. Additional Immune Checkpoint Inhibitors

In certain aspects, the immune checkpoint inhibitor is a CD80 antagonist, a CD86 antagonist, a TIM-3 antagonist, a TIGIT antagonist, a CD20 antagonist, a CD96 antagonist, a IDO1 antagonist, a STING antagonist, a GARP antagonist, a CD40 antagonist, A2aR antagonist, a CEACAMI (CD66a) antagonist, a CEA antagonist, a CD47 antagonist a PVRIG antagonist, a TDO antagonist, a VISTA antagonist, or a KIR antagonist.

In one aspect, the immune checkpoint inhibitor is a KIR antagonist. In certain aspects, the KIR antagonist is an anti-KIR antibody or antigen binding fragment thereof. In some aspects, the anti-KIR antibody is lirilumab (1-7F9, BMS-986015, IPH 2101) or IPH4102.

In one aspect, the immune checkpoint inhibitor is TIGIT antagonist. In one aspect, the TIGIT antagonist is an anti-TIGIT antibody or antigen binding fragment thereof. In certain aspects, the anti-TIGIT antibody is BMS-986207, AB 154, COM902 (CGEN-15137), or OMP-313M32.

In one aspect, the immune checkpoint inhibitor is a TIM-3 antagonist. In certain aspects, the TIM-3 antagonist is an anti-TIM-3 antibody or antigen binding fragment thereof. In some aspects, the anti-TIM-3 antibody is TSR-022 or LY3321367.

In one aspect, the immune checkpoint inhibitor is an IDO1 antagonist. In another aspect, the IDO1 antagonist is indoximod (NLG8189; 1-methyl-D-TRP), epacadostat (INCB-024360, INCB-24360), KHK2455, PF-06840003, navoximod (RG6078, GDC-0919, NLG919), BMS-986205 (F001287), or pyrrolidine-2,5-dione derivatives.

In one aspect, the immune checkpoint inhibitor is a STING antagonist. In certain aspects, the STING antagonist is 2′ or 3′-mono-fluoro substituted cyclic-di-nucleotides; 2′3′-di-fluoro substituted mixed linkage 2′,5′-3′,5′ cyclic-di-nucleotides; 2′-fluoro substituted, bis-3′,5′ cyclic-di-nucleotides; 2′,2″-diF-Rp,Rp,bis-3′,5′ cyclic-di-nucleotides; or fluorinated cyclic-di-nucleotides.

In one aspect, the immune checkpoint inhibitor is CD20 antagonist. In some aspects, the CD20 antagonist is an anti-CD20 antibody or antigen binding fragment thereof. In one aspect, the anti-CD20 antibody is rituximab (RITUXAN; IDEC-102; IDEC-C2B8), ABP 798, ofatumumab, or obinutuzumab.

In one aspect, the immune checkpoint inhibitor is CD80 antagonist. In certain aspects, the CD80 antagonist is an anti-CD80 antibody or antigen binding fragment thereof. In one aspect, the anti-CD80 antibody is galiximab or AV 1142742.

In one aspect, the immune checkpoint inhibitor is a GARP antagonist. In some aspects, the GARP antagonist is an anti-GARP antibody or antigen binding fragment thereof. In certain aspects, the anti-GARP antibody is ARGX-115.

In one aspect, the immune checkpoint inhibitor is a CD40 antagonist. In certain aspects, the CD40 antagonist is an anti-CD40 antibody for antigen binding fragment thereof. In some aspects, the anti-CD40 antibody is BMS3h-56, lucatumumab (HCD122 and CHIR-12.12), CHIR-5.9, or dacetuzumab (huS2C6, PRO 64553, RG 3636, SGN 14, SGN-40). In another aspect, the CD40 antagonist is a soluble CD40 ligand (CD40-L). In one aspect, the soluble CD40 ligand is a fusion polypeptide. In one aspect, the soluble CD40 ligand is a CD40-L/FC2 or a monomeric CD40-L.

In one aspect, the immune checkpoint inhibitor is an A2aR antagonist. In some aspects, the A2aR antagonist is a small molecule. In certain aspects, the A2aR antagonist is CPI-444, PBF-509, istradefylline (KW-6002), preladenant (SCH420814), tozadenant (SYN115), vipadenant (BIIB014), HTL-1071, ST1535, SCH412348, SCH442416, SCH58261, ZM241385, or AZD4635.

In one aspect, the immune checkpoint inhibitor is a CEACAMI antagonist. In some aspects, the CEACAMI antagonist is an anti-CEACAMI antibody or antigen binding fragment thereof. In one aspect, the anti-CEACAMI antibody is CM-24 (MK-6018).

In one aspect, the immune checkpoint inhibitor is a CEA antagonist. In one aspect, the CEA antagonist is an anti-CEA antibody or antigen binding fragment thereof. In certain aspects, the anti-CEA antibody is cergutuzumab amunaleukin (RG7813, RO-6895882) or RG7802 (RO6958688).

In one aspect, the immune checkpoint inhibitor is a CD47 antagonist. In some aspects, the CD47 antagonist is an anti-CD47 antibody or antigen binding fragment thereof. In certain aspects, the anti-CD47 antibody is HuF9-G4, CC-90002, TTI-621, ALX148, NI-1701, NI-1801, SRF231, or Effi-DEM.

In one aspect, the immune checkpoint inhibitor is a PVRIG antagonist. In certain aspects, the PVRIG antagonist is an anti-PVRIG antibody or antigen binding fragment thereof. In one aspect, the anti-PVRIG antibody is COM701 (CGEN-15029).

In one aspect, the immune checkpoint inhibitor is a TDO antagonist. In one aspect, the TDO antagonist is a 4-(indol-3-yl)-pyrazole derivative, a 3-indol substituted derivative, or a 3-(indol-3-yl)-pyridine derivative. In another aspect, the immune checkpoint inhibitor is a dual IDO and TDO antagonist. In one aspect, the dual IDO and TDO antagonist is a small molecule.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the agonist of an immune checkpoint molecule is an agonist of OX40, CD27, CD28, GITR, ICOS, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

### 7. Patient Populations

Provided herein are clinical methods for treating tumors in subjects, e.g., human patients, using a therapy disclosed herein, for example, a T-cell checkpoint inhibitor (e.g., an anti-PD-L1 antibody), a CD73 inhibitor (e.g., an anti-CD73 antibody), and chemotherapy and/or radiotherapy.

Examples of cancers and/or malignant tumors that may be treated using the methods of the disclosure, include liver cancer, hepatocellular carcinoma (HCC), bone cancer, pancreatic cancer, skin cancer, oral cancer, cancer of the head or neck, breast cancer, lung cancer, small cell lung cancer, NSCLC, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, squamous cell carcinoma of the head and neck (SCCHN), non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present disclosure is also applicable to treatment of metastatic cancers. In aspects, the cancer is renal cell carcinoma (RCC), gastric/gastoesophogeal junction carcinoma, non-small cell lung carcinoma (NSCLC), melanoma, squamous cell carcinoma of the head and neck (SCCHN), hepatocellular carcinoma, or urothelial carcinoma.

In one aspect, the human patient suffers from a malignant tumor that is refractory to treatment with an immune checkpoint inhibitor. In another aspect, the patient suffers from a malignant tumor that is refractory to treatment with a PD-L1 inhibitor. In another aspect, the patient suffers from a malignant tumor that is refractory to treatment with an anti-PD-L1 antibody.

### EXAMPLES

### Experimental Methods

### Animal Studies

In vivo studies were performed using 8-10 week old BALB/cAnNCrl mice (Charles River UK) or C57BL/6. Mice were housed in an AstraZeneca vivarium with access to food and water ad libidum and were cared for daily by trained personnel. Mice were acclimatized to the vivarium conditions for a week and handled according to the Home Office Animals Scientific Procedures Act, 1986, UK. All animal work was carried out under a Home Office approved project licence (PPL P49077891) and U.S. Plant Pat. No. 3,208,003 (RTx studies) as well as according to the institutional guidelines. BALB/c mice were subcutaneously implanted with 0.5e6 CT26 (colorectal) tumor cells. C57BL/6 mice were subcutaneously implanted with 0.5e6 MCA205 (fibrosarcoma) tumor cells in 50% Matrigel® (Corning, Cat. No. 356231) in PBS or with 0.5e6 MC38 cells in PBS. Tumors were measured with callipers three times a week, starting from day-5. Tumor bearing mice were treated either with combinations of oxaliplatin (Hospira, Clinical grade, 5 mg/mL) at 6 mg/Kg dose level and 5-Fluorouracil (Hospira, Clinical grade, 50 mg/mL) at 50 mg/Kg dose level or docetaxel (Accord, Clinical grade, 20 mg/mL) dosed at 10 mg/Kg along with murine surrogate monoclonal antibodies for Oleclumab (clone 10.3, an anti-CD73 with murine IgG1 Fc sequence, 10 mg/Kg or 20 mg/Kg) and Durvalumab (clone 80, a chimeric rat anti-mouse PD-L1 antibody with IgG1 Fc sequence, 10 mg/Kg). The dose and schedule of chemotherapy employed were based on previously published information (Dosset M et al., Oncoimmunology 2018; 7; Gao Q et al., J Immunother Cancer 2019; 7:42). In an attempt to define contribution of components, comparator groups received monotherapies and other iterations of the combination. Groups of mice were removed for pharmacodynamic analysis MSI, and transcriptomics) as well as immunohistochemical (IHC) analysis at appropriate timepoints. For the radiotherapy experiments, tumors were treated with five doses of 2Gy fraction given on each consecutive day using X-ray source Self-Contained Cabinet Irradiator (Xstrahl CIX3).

In vitro Assays

Cells (HCT-116, HT-29, CT-26 and MCA-205) are seeded in 96-well culture plates at 1e⁴ cells/well in 40 μl of complete culture media and cultured in a 37° C./5% CO₂ incubator for about 4 hours to allow cell adherence. 10 μl/well of Oleclumab (5 nM) at 5× final concentration is added to the cells and cultured in a 37° C./5% CO₂ incubator overnight to allow Oleclumab pre-treatment. 50 μl/well of serially diluted chemotherapeutic drugs (5-Fluorouracil, Oxaliplatin, Docetaxel) at 2× final concentrations are added to the cells, in accordance to the treatment groups and cultured in a 37° C./5% CO₂ incubator for 72 h. Cell viability was measured using CellTiter-Glo® Luminescent assay (Promega, Cat. No. G7573).

### RNA Preparation for Bulk Sequencing

Tumor tissue was flash-frozen in liquid nitrogen and stored at −80° C. at the time of collection in the animal unit and was sent to Novogene on dry ice. Tissue processing and RNA extraction was carried out by Novogene Co. by using the QIAGEN RNeasy Plus Universal kit (Qiagen, Cat. No. 73404), according to the manufacturer's protocol. Briefly, tissue samples were homogenized in QIAzol Lysis Reagent. The homogenate was then separated into aqueous and organic phases by centrifugation after addition of gDNA eliminator solution and chloroform. The upper, aqueous phase containing RNA was collected, and RNA purified using RNeasy spin columns. RNA quality check (QC) was performed by 1% Agarose gel electrophoresis, amount and purity were measured by Nanodrop, and RNA Integrating Number (RIN) was obtained by Bioanalyzer Agilent2100.

NEBNextR Ultra RNA library prep kit (Cat. No. E7530L) was used for library preparation for sequencing. Samples were sequenced using Illumina PE150 (50M reads/sample) bulk RNA-seq and downstream bioinformatic analysis was performed.

### Analysis of RNASeq Data

Reads were mapped to Mus musculus genome (mm10) genome using Star (Dobin A et al., Bioinformatics 2013; 29:15-21). Uniquely mapped reads were counted using htseq-count (Putri GH et al., Bioinformatics 2022; 38:2943-5). DESeq2 (Love M I et al., Genome Biol 2014; 15) was used to normalize counts with size factors and identify differentially expressed genes across conditions with a threshold of abs (log2FC)>1 and adjusted p value<0.05.

Gene set enrichment analysis was performed using R package ‘fGSEA’ (Korotkevich G et al., bioRxiv 2021; 060012) using hallmark gene sets from mouse MSigDB (Subramanian A. et al., Proc Natl Acad Sci USA 2005; 102:15545-50). Enrichment p-values were calculated as described in Korotkevich G et al., bioRxiv 2021; 060012 and p-values were adjusted using Benjamini-Hochberg method. Gene ontology Biological Process enrichments were identified using R package topGO (Bioconductor-topGO) with adj-pval<0.05. KEGG pathway enrichment analysis was conducted using R package clusterProfiler (Wu T. et al., The Innovation 2021; 2:100141). MCPCounter tool (Becht E. et al., Genome Biol 2016; 17:1-20) was used to estimate immune cell abundances for each sample and condition using immune gene signatures within MCPCounter.

### Tissue Preparation for Mass Spectrometry Imaging (MSI)

Tumors were snap frozen in liquid nitrogen immediately after resection and the frozen tissues were embedded in a HPMC/PVP hydrogel as previously described (Dannhorn A. et al., Anal Chem 2020; 92:11080-8). Sectioning was performed on a CM3050 S cryostat (Leica Biosystems, Nussloch, Germany) at a section thickness of 10 um and the tissue sections were immediately thaw mounted and dried under a stream of nitrogen and sealed in vacuum pouches to preserve the metabolic integrity of the sections. Tissue sections for DESI-MSI and IMC were thaw-mounted onto Superfrost microscope slides (VWR, Cat. No. 630-2863), whilst sections prepared for MALDI-MSI were thaw mounted onto conductive indium tin oxide (ITO) coated slides (Bruker Daltonik, Cat. No. 8237001). Polyvinylpyrrolidone (PVP) and (Hydroxypropyl) methylcellulose (HPMC) (purchased from Merck (Cat. No.PVP360; H8384). Methanol (Cat. No. 15624680), iso-pentane (Cat. No. 15692830) and isopropyl alcohol (Cat. No. 10674732) were obtained from Fisher Scientific.

### Mass Spectrometry Imaging (MSI)

DESI-MSI analysis was performed on a Q-Exactive mass spectrometer (Thermo Scientific, Bremen, Germany) equipped with an automated 2D-DESI ion source (Prosolia Inc., Indianapolis, IN, USA) operated in negative ion mode, covering the applicable mass range up to m/z of 1000, with a nominal mass resolution of 70,000. The injection time was fixed to 150 ms resulting in a scan rate of 3.8 pixel/s. The spatial resolution was set to 70 μm. A home-built Swagelok DESI sprayer was operated with a mixture of 95% methanol, 5% water delivered with a flow rate of 272 1.5 μL/min and nebulized with nitrogen at a backpressure of 6 bar. The resulting raw files were converted into.mzML files using ProteoWizard msConvert (version 3.0.4043) and 274 subsequently compiled to an.imzML file (imzML converter version 1.3) (Race A M et al., J Proteomics 2012; 75:5111-2). All subsequent data processing was performed in SCILS Lab (version 2021b, Bruker Daltonik, Bremen, Germany).

MALDI-MSI analysis was performed on a RapifleX Tissuetyper instrument (Bruker Daltonik, Bremen, Germany) operated in negative detection mode. 9-Aminoacridine (9-AA) prepared in 80:20 methanol: water was used as a MALDI matrix and spray deposited using an automated spray system (M3-Sprayer, HTX technologies, Chapel Hill, NC, USA). MALDI experiments were performed with a spatial resolution of 50 μm. A total of 400 laser shots were summed up per pixel to give the final spectra. For all experiments, the laser was operated with a repetition rate of 10 kHz. All raw data was directly uploaded and processed in SCILS lab (Version 2021b) software packages. All DESI and MALDI data and images were normalised to the total ion current (TIC) to compensate for signal variation across the course of the experiments. The data analysis performed in the SCiLS lab software packages included classification of the datasets based on manual annotation of the MSI data guided by the histology to identify “tumor”, and “necrosis” while removing the background as applicable. The tissue classification was performed using partial least squares-discriminant analysis (PLS-DA). The “necrotic margin” was delineated and differentiated from the “viable tumor” by subjecting the “tumor” tissue cluster to a pixel-wise principal component analysis (PCA). The loadings of principal components (PCs) highlighting the tissue compartment adjacent to necrotic areas were extracted and used to create a peak list for unsupervised segmentation based on a bisecting K-means classifier. All data shown was extracted from the “viable tumor” cluster.

### Imaging Mass Cytometry (IMC)

Imaging mass cytometry was performed on a slide which had been analysed by DESI-MSI. Antibodies used for IMC staining are shown Table 1.

| Label | Clone | Channel | Dilution | Vendor | Product Code |
|---|---|---|---|---|---|
| αSMA | Polyclonal | Pr(141) | 1:100 | Standard BioTools | 3141017D |
| Vimentin | D21H3 | Nd(143) | 1:100 | Standard BioTools | 3143027D |
| CD68 | FA-11 | Nd(145) | 1:100 | Bio Rad antibodies | MCA1957GA |
| F4/80 | CI:A3-1 | Gd(155) | 1:100 | Cell signalling | #70076 |
| CD163 | TNKUPJ | Gd(156) | 1:50 | Thermo Fisher | 14-1631-82 |
| CD31 | 390 | Dy(164) | 1:100 | Thermo Fisher | 14-0311-82 |
| CD206 | CD68C2 | Tm(169) | 1:50 | Standard BioTools | 3169021B |
| DNA | Intercalator | Ir(191) | 1:400 | Standard BioTools | 201192A |
| CollagenIV | Polyclonal | Bi(209) | 1:100 | Novotec Labs | 20451 |

Antibodies were either purchased pre-conjugated with a heavy-metal tag and untagged antibodies were conjugated in house, using Fluidigm Maxpar Antibody Labelling Kit, according to manufacturer's instructions. The slides were fixed with 4% paraformaldehyde in phosphate-buffered saline (PBS) for 10 minutes. The slides were washed 3×5 minutes in PBS, permeabilized for 5 minutes with 1:1000 dilution of Triton X-100 in Casein Solution, washed 3×5 minutes in PBS, and blocked for 30 minutes with Casein Solution. Antibodies were diluted to an appropriate concentration and the slides incubated overnight with the antibody solution at 4° C. The slides were washed 3×5 minutes in PBS and nuclei were stained with DNA Intercalator-iridium at a dilution of 1:400 in PBS for 30 minutes. The slides were washed 3×5 minutes in PBS, 30 seconds in deionized water, then dried for storage at room temperature until analysis. A region for IMC analysis was selected using consecutive H&E stained sections and the MSI results. Areas of approximately 1.5×1.5 to 2.0×2.0 mm were selected for analysis to include necrotic, necrotic margin and viable tumor regions. IMC analysis was performed using a Hyperion instrument (Fluidigm Corporation, San Francisco, CA, USA) with an ablation energy of 4 db an ablation frequency of 100 Hz.

IMC data were analysed using Halo v.3.3.2541.366 (Indica Laboratories, Albuquerque, NM, USA). Tissue regions were classified into viable tumor, necrosis, necrotic margin and off-tissue using Random Forest. Analysis was performed using Highplex FL v4.0.4. cell segmentation and thresholds were optimized manually. Nuclear segmentation was performed using 193Ir DNA intercalator channel. IMC images were adjusted and extracted using the build-in figure maker tool.

### Immunohistochemistry

A portion of each tumor was immersion-fixed in 10% neutral-buffered formalin, then processed to paraffin using routine methods. Tissues were sectioned at 4 mm thickness and immunohistochemically stained using a rabbit monoclonal antibody to CD73 (D7F9A, Cell Signaling Technology) at a 0.5 μg/ml dilution, on an automated Leica Bond-RX immunostainer, using DAB as a chromogen and hematoxylin as background stain. This antibody was previously shown not to have binding inhibition in tissues previously treated with Oleclumab (unpublished observations).

Resulting slides were digitally scanned at 20× magnification using an Aperio scanner (Leica Biosystems). For analysis, measurement of positive CD73 area and of hematoxylin-positive area were performed using Halo image analysis software (Indica Labs). Briefly, the tumors areas were manually annotated, and the positive DAB and hematoxylin areas were measured in the annotated area using the Halo area quantification algorithm, adapted to the staining characteristics of the tissues. Percent of CD73-positive area and hematoxylin-positive areas within the total tumor area were obtained. The final data output used was the ratio of CD73-to-hematoxylin areas, hematoxylin area being considered a representative indicator of cellularity (as it mainly stains nuclei), allowing normalisation of CD73-positive area to the overall cellularity of the tumors.

Statistical Analysis

All in vivo data were collated in the Excel spreadsheets and transferred to GraphPad Prism 9.00 (GraphPad Software Inc.) for graphical representation and statistical analysis. After outlier identification and Shapiro-Wilk test for normality distribution, statistical significance was determined by One-way ANOVA followed by Dunnett's multiple comparison for comparisons of more than two groups if data were normally distributed. Kruskal-Wallis Test with Dunn's multiple comparisons test was used if data were not normally distributed, as detailed in figure legends. Survival studies were analysed with a log rank (Mantel-Cox) test, comparing only two survival curves at a time. P values were not adjusted for multiple testing.

EXAMPLE 1: Combined anti-CD73, anti-PD-L1 antibodies, and 5FU+OHP treatment leads to enhanced complete responses in syngeneic mouse models

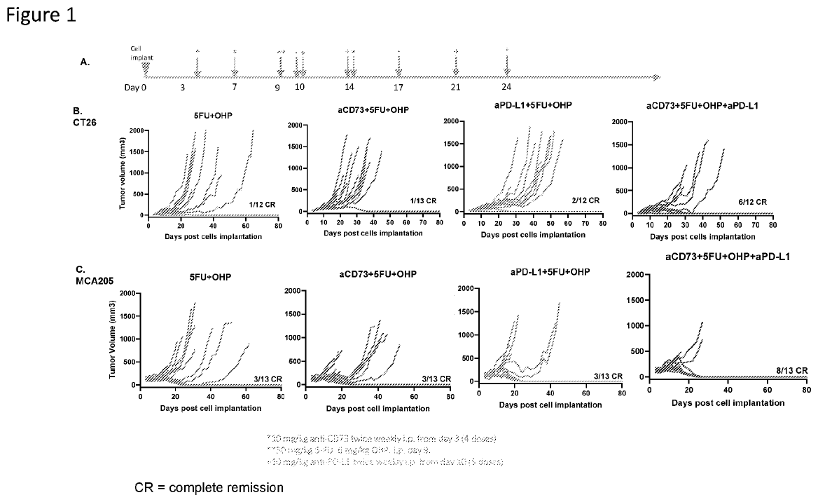

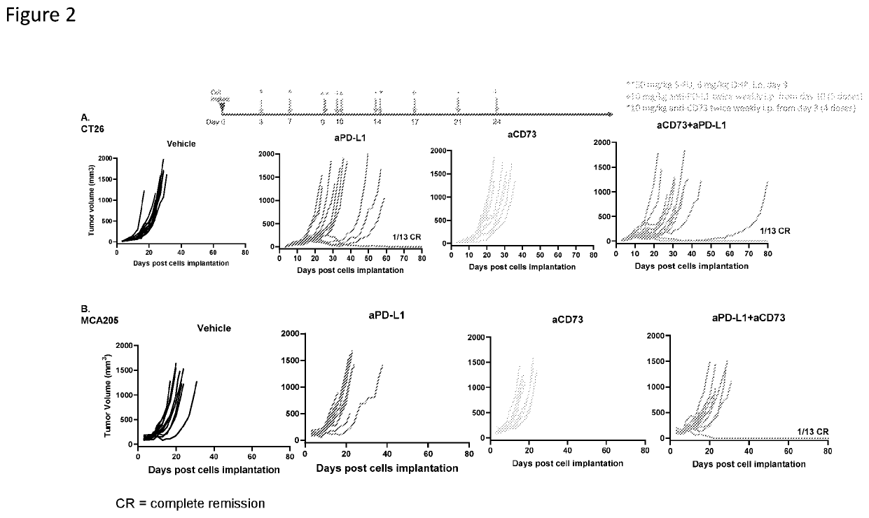

Combined treatment with anti-CD73 (aCD73) and anti-PD-L1 (aPD-L1) antibodies, in addition to 5-fluorouracil (5FU)+oxaliplatin (OHP) resulted in enhanced efficacy and complete responses in two mouse syngeneic models of cancer-CT26 (p=0.005) and MCA205 (p=0.008) as shown in FIG. 1B and 1C (Kruskal-Wallis test). Treatment of CT26 or MCA205 tumor bearing mice with aCD73 as a monotherapy resulted in negligible benefit in terms of tumor growth inhibition. Anti-PD-L1 treatment retarded tumor growth rates in a proportion of mice bearing CT26 tumors, but displayed minimal activity in the MCA205 model (FIG. 2). 5FU+OHP chemotherapy supressed tumor growth rates in both the models, but combining 5FU+OHP with either immune-oncology (IO) agents (anti-CD73 or anti-PD-L1) exerted minimal further uplift in efficacy. A combination treatment utilizing anti-CD73 and anti-PD-L1 antibodies (aCD73+aPD-L1) afforded similar levels of tumor growth control to anti-PD-L1 alone, highlighting the importance of the chemotherapy component within the chemotherapy+IO combination (FIGS. 1 and 2).

### Example 2

### Effect of aCD73 on Chemotherapy Induced Cytotoxicity in Cell Culture

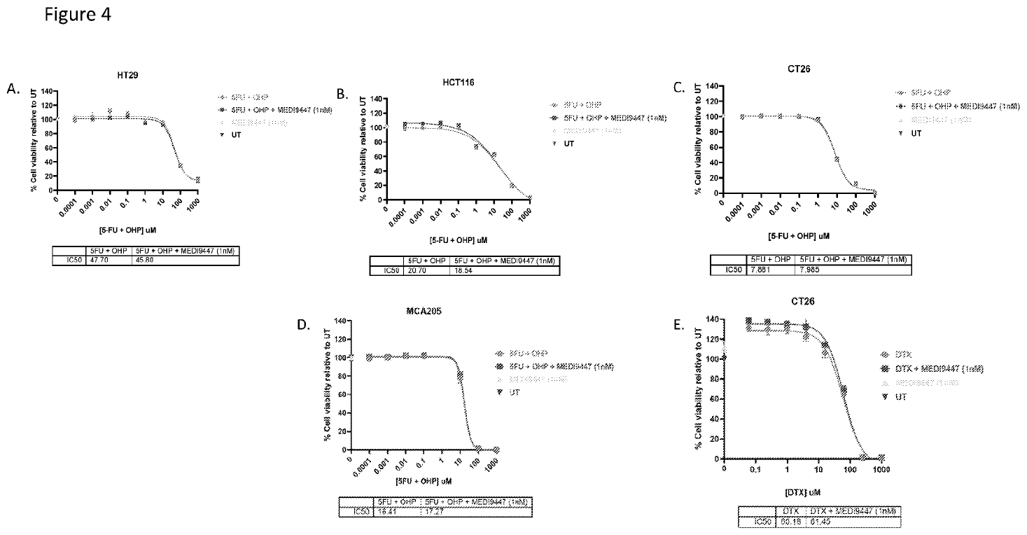

In parallel, whether the effects seen in vivo were tumor microenvironment (TME) driven or not, were investigated by determining whether anti-CD73 could enhance the direct cytotoxic effect of 5FU+OHP on the cell lines themselves. However, no evidence was found that this was occurring in the in vitro setting (FIG. 4). Whether the cytotoxic effects of docetaxel on cultured CT26 cells was influenced by the presence of aCD73 was also investigated. No influence of the antibody was detected.

### Example 3

### CD8 Cell Depletion Compromises Efficacy of the Quadruple Combination (aCD73+aPD-L1+5FU+OHP) in MCA205 Tumor Bearing Mice

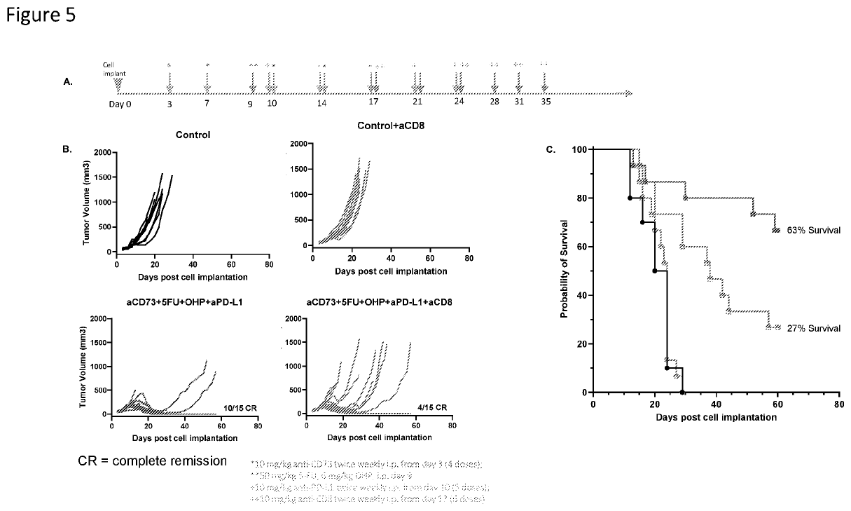

To test the hypothesis that CD8 T-cells played a part in the enhanced efficacy of the combination, CD8 T cells in the MCA205 model were selectively depleted, using IP injection of clone 53-6.7 from the 17th day post tumor implantation (schematic FIG. 5A). This experiment confirmed significantly diminished tumor control and long term survival (by >50%) in the combination (5FU+OHP+aCD73+aPD-L1) treated group (Log rank test, p=0.026, FIG. 5C). These CD8 depletion data suggest cell-mediated immunity plays an essential role in the observed activity of the chemotherapy-IO combination. This does not rule out contribution by other immune-system components or ‘conventional’ cytotoxic or cytostatic effects mediated by 5FU+OHP.

### Example 4

### ‘Early’ Pharmacodynamic Effect of aCD73+5FU+OHP With Respect to CD73 Expression, Adenosine Pathway Metabolites and Stromal Cell Populations in the Murine CT26 Tumor Model

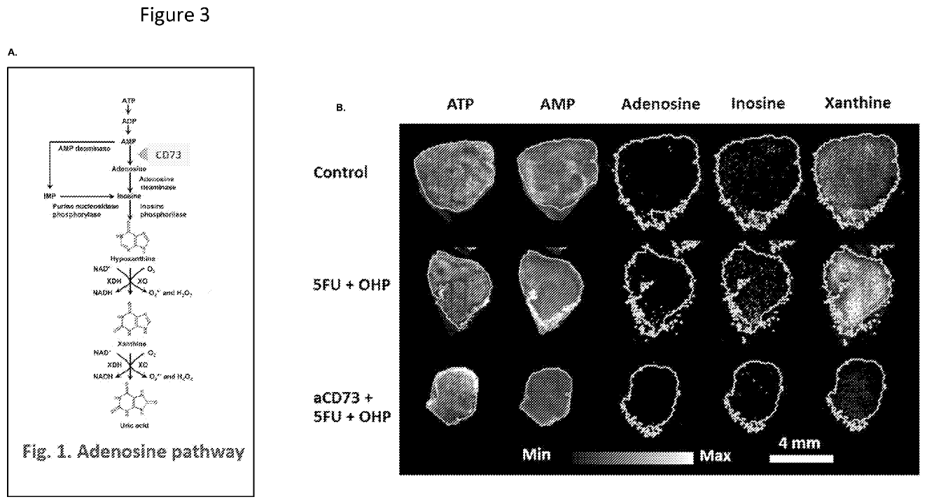

Pharmacodynamic activity of the aCD73 and aPD-LI plus 5FU+OHP chemotherapy combination, was evaluated using Immunohistochemistry (IHC) and Imaging Mass Cytometry (IMC) to understand the early changes mediated by CD73 blockade. As anticipated, the murine surrogate antibody of Oleclumab lowered CD73 levels in CT26 tumors. The IHC based detection of CD73 employed an antibody that did not compete with the murine surrogate antibody of Oleclumab; therefore the lower levels of CD73 protein were consistent with Oleclumab's known ability to internalize CD73. IMC highlighted that CT26 tumors from aCD73 treated mice tended to have lower frequencies of cells expressing markers known to associate with cancer associated fibroblasts and with the suppressive tumor associated macrophages. Mass Spectroscopy Imaging (MSI) revealed a notable trend for adenosine, inosine and xanthine suppression in CT26 tumors from mice treated with anti-CD73 (FIG. 3 and FIG. 5). Conversely adenosine monophosphate (AMP), the main substate of CD73, appeared elevated relative to other treatment groups, in tumor tissue from mice treated with chemotherapy and the anti-CD73 combination. These pharmacodynamic data are consistent with Oleculmab's proposed mechanism of action; in terms of CD73 targeting, enzymatic inhibition and adenosine pathway modulation.

### Example 5

### Pharmacodynamic Effect of aCD73+aPD-L1+5FU+OHP With Respect to CT26 Transcriptome

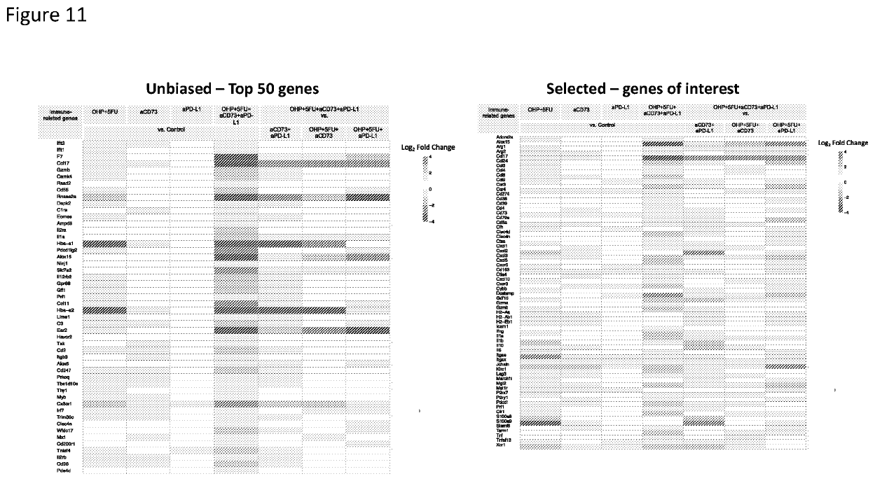

To further gain a deeper understanding of the mechanistic basis for the enhanced activity in the quadruple group, RNAseq was used to explore changes within the CT26 transcriptome. Using DEseq2 and fGSEA packages, minimal changes in the CT26 transcriptome were identified following aCD73 or aPD-LI treatment as monotherapies (See, Table 2 below). 5FU+OHP, on the other hand, was highly perturbing; causing significant upregulation of 277 genes and downregulation of a further 158, relative to control tumors. Gene Ontology (GO BP) base pair enrichments and KEGG pathway analysis of these data highlighted significant effects on genes associated with immune response, leukocyte, NK and T-cell activation, T cell receptor signaling, and Interferon (Type 1 & 2) production (adjusted-pval<0.05). Individual genes upregulated in line with this included: CCL3, 4, 8, 17, CXCL10, GDF15, CD8a, IFNγ, Perforin, Granzyme, Lag-3 and PD-1. CXCL2 expression was reduced (FIG. 11). These RNAseq data highlight that 5FU+OHP treatment exerts significant perturbance of genes associated with immune-functionality in the murine CT26 mouse model of cancer.

In contrast to the minor transcriptomic changes observed with the constituent monotherapy treatments, the combination of aCD73 and aPD-L1 resulted in 1236 differentially expressed genes. Most significant and wide-ranging transcriptomic changes were achieved with the 5FU+OHP+aCD73+aPD-L1 combination; causing 1490 genes to be upregulated and 128 down regulated in comparison to untreated tumors (Table 2).

| | | Gene Ontology (BP) | |
|---|---|---|---|
| | | enrichments | KEGG pathway enrichments |
| | | (adjusted-pval <0.05) | (adjusted-pval <0.05) |
| aPDL1 | No DE | — | — |
| | genes | | |
| aCD73 | 1 DE | — | — |
| | genes: 1 | | |
| | Up- | | |
| | regulated | | |
| FF | 435 DE | Immune response, | Natural killer cell mediated cytotoxicity |
| | genes: | Lymphocyte | T cell receptor signaling pathway |
| | 277 Up- | activation, Leukocyte | Th1 and Th2 cell differentiation |
| | regulated | activation, T cell | Cytokine-cytokine receptor interaction |
| | & | activation, Interferon- | |
| | 158 | gamma production | |
| | Down- | | |
| | regulated | | |
| aCD73. | 1236 DE | Inflammatory | Cytokine-cytokine receptor interaction |
| aPDL1 | genes: | response, Myeloid | Hematopoietic cell lineage |
| | 1049 Up- | leukocyte migration, | Chemokine signaling pathway |
| | regulated | Cell chemotaxis | Complement and coagulation cascades |
| | & | | |
| | 187 | | |
| | Down- | | |
| | regulated | | |
| FF.aCD | 964 DE | Immune response, | Cytokine-cytokine receptor interaction |
| 73 | genes: | Lymphocyte | Hematopoietic cell lineage |
| | 902 Up- | activation, Leukocyte | Chemokine signaling pathway |
| | regulated | activation, T cell | T cell receptor signaling pathway |
| | & | activation, Interferon- | Th1 and Th2 cell differentiation |
| | 62 | gamma production | NF-kappa B signaling pathway |
| | Down- | | Complement and coagulation cascades |
| | regulated | | |
| FF.aPD | 457 DE | Immune response, | T cell receptor signaling pathway |
| L1 | genes: | Lymphocyte | Cytokine-cytokine receptor interaction |
| | 333 Up- | activation, Leukocyte | Th1 and Th2 cell differentiation |
| | regulated | activation, T cell | Natural killer cell mediated cytotoxicity |
| | & | activation, Interferon- | |
| | | gamma production | |
| | 124 | | |
| | Down- | | |
| | regulated | | |
| FF.aCD | 1618 DE | Immune response, | T cell receptor signaling pathway, |
| 73.aPD | genes: | Leukocyte activation, | Th1 and Th2 cell differentiation, |
| L1 | 1490 Up- | T cell activation, | Complement and coagulation cascades, |
| | regulated | Lymphocyte activation | Chemokine signaling pathway, |
| | & | | Natural killer cell mediated cytotoxicity |
| | 128 | | |
| | Down- | | |
| | regulated | | |

KEGG pathway and Gene Ontology enrichments analysis highlighted chemotactic mobilization, T cell activation, T cell receptor signaling, Th1 and Th2 cell differentiation and Natural killer cell mediated cytotoxicity. The most affected immune-related genes are listed in FIG. 11. There were several notable additions, to the genes upregulated by 5FU+OHP treatment, including (but not limited to): CD38, CD39, CXCL1, CXCL3, CXCL5, CD163, CTLA4, CXCR3, Granzyme A, ICAM1, 116, P2RY1, TNF-a, IL2a, Il1a, ALOX15, SLC7a2, Ear2, Havc2.

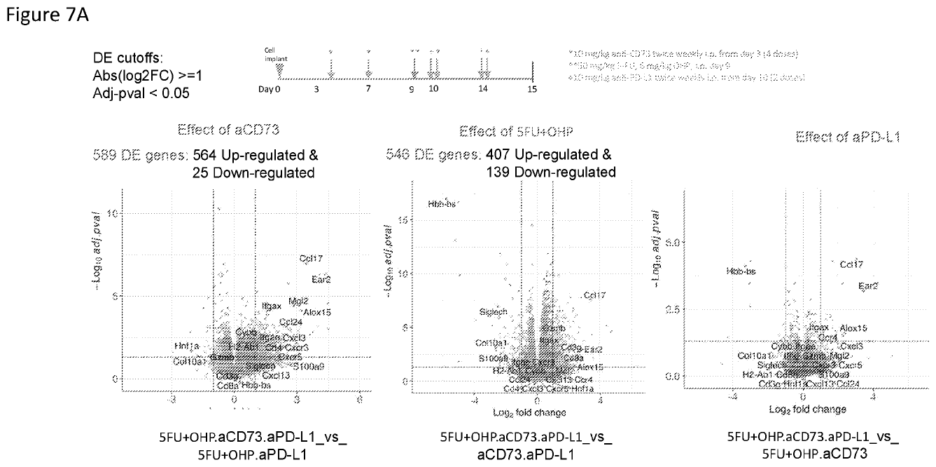

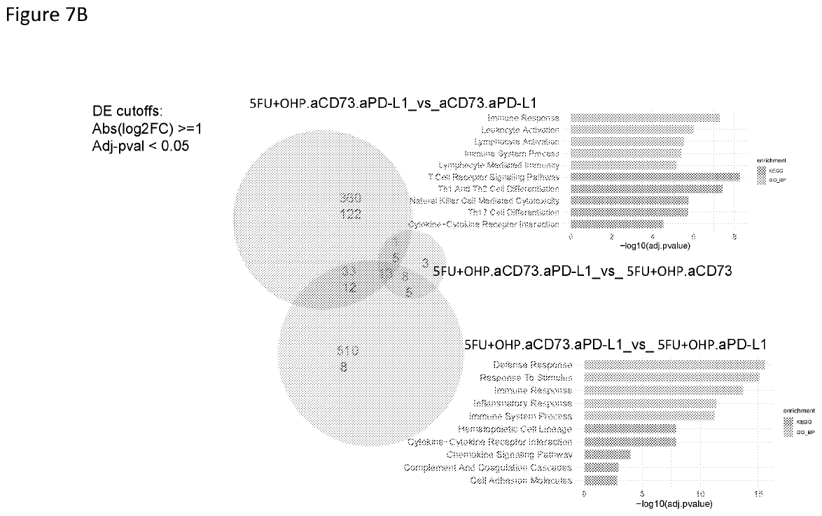

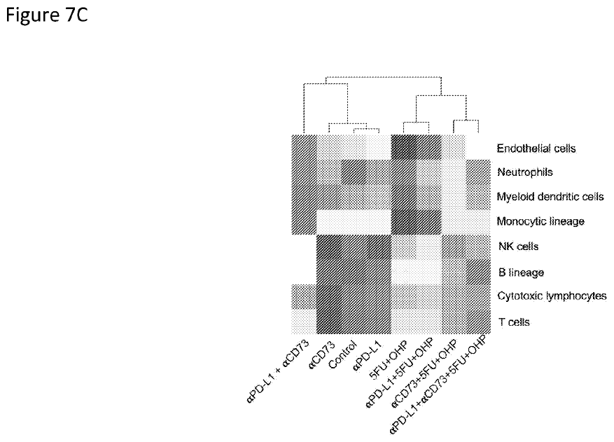

Inclusion of treatment groups representing the various components of the combination allowed deconvolution of the contribution of the individual therapeutic components (FIG. 7A, 7B and 11, Table 2). This analysis highlighted a critical role for 5FU+OHP in driving interferon pathway (type 1 and 2) activation alongside T-cell and NK cell activation, cytotoxic activity and IL2/STAT5 pathway signaling. The effects of 5FU+OHP inclusion were largely differentiated from those mediated by addition of either of the IO drug components to the treatment, as evidenced the 360 genes uniquely upregulated and 122 downregulated by the 5FU+OHP component. Including 5FU+OHP within the combined aCD73+aPD-L1 treatment approach, served to elevate expression of key immune-related genes such as Interferon-gamma, TRIM6, CCL17, Granzyme B, GDF15, perforin and Lag3. Conversely, IL-10, IL-1b, CXCL2, S100A8 were downregulated when 5FU+OHP was included.

Adding CD73 blockade to the combination of 5FU+OHP and aPD-L1 upregulated 510 genes and downregulated 8, that were not modulated within other iterations of the combination as shown in FIG. 7A. Notably influenced genes included CXCR3, H2-AB1, Itgae, CXCL3, Mgl2, CXCR5, CD4 and Cybb. This also drove even higher expression of CCL17, a major tumor infiltrating lymphocyte (TIL) attracting chemokine, and CCL24 a chemokine known to preferentially chemoattract MI macrophages (Xuan W, et al., J Leukoc Biol 2015; 97:61-9). These data highlight a novel and impactful role for adenosine pathway inhibition within chemotherapy/checkpoint inhibitor combinations. Particularly with respect to individual genes and signatures linked to myeloid and B cell biology. CD38 and P2Y1, two genes known to be linked to the adenosine pathway itself, were also significantly upregulated when the chemotherapy plus anti-PD-L1 group was augmented with anti-CD73. Withholding aPD-L1 from the 5FU+OHP plus aCD73 combination treatment was detrimental in terms of upregulating genes related to inflammation, immune-response, and interferon gamma pathway activation. Notably, ALOX15, a gene linked to macrophage function and efferocytosis, and IL-1b were affected.

Next we checked if the gene perturbation at this scale also led to changes in the enumeration of the cell populations. For this we estimated the abundance of immune cell populations in the treated CT26 tumors, using the MCP-Counter tool (Becht E, et al., Genome Biol 2016; 17:1-20). This computational analysis highlighted a prominent effect of oxaliplatin and 5-fluorouracil chemotherapy, in terms of increased lymphocyte representation; in line with significantly elevated proinflammatory and chemotactic chemokine/cytokine gene expression levels following this treatment (FIG. 7C). CT26 tumors from mice treated with monotherapies and combinations excluding the chemotherapy component were notably less infiltrated with lymphocytes. The 5FU+OHP+aCD73+aPD-L1 treatment stood out prominently for the elevated tumoral abundances of both lymphocytes (cytotoxic T-cell, NK, B-cells) and myeloid cell populations (including monocytic dendritic cells); a profile conducive to improved tumor control and increased overall survival times in cancer models (Petitprez F, et al., Nature 2020 577:7791; Voss M H, et al., JCO20203815_suppl5025 2020; 38:5025-5025; Chambers A M, et al., Front Immunol 2018; 9:2533; Mastelic-Gavillet B, et al., J Immunother Cancer 2019; 7:1-16).

### Example 6

### Combined aCD73, aPD-L1 and Docetaxel Treatment Leads to Enhanced Complete Responses in CT26 Syngeneic Mouse Model

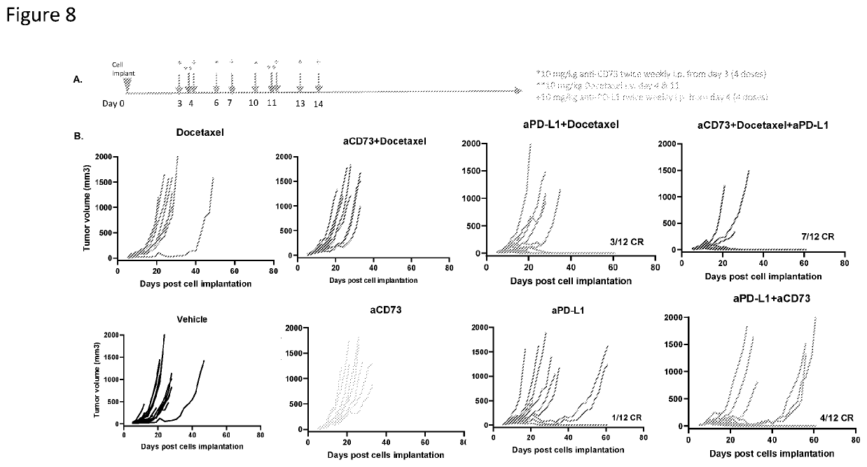

Importantly, the synergistic combinatorial effects noted with 5FU+OHP were found to extend to a second chemotherapy class; i.e. Docetaxel (DTX). The combination approach was overlayed onto an already established tolerated dosing scheme for this chemotherapy. In this case the combination of DTX, aPD-L1 and aCD73 resulted in significantly improved tumor growth-inhibition and 7 out of 12 (58%) complete responses (p=0.0001) compared to maximum of 3 out of 12 (25%) in the aPD-LI plus docetaxel combination group (p=0.001), Kruskal-Wallis test, as shown in FIG. 8.

### EXAMPLE 7

### Fractionated Radiotherapy is Enhanced by Combined aCD73, aPD-L1 in MC38 Syngeneic Mouse Model

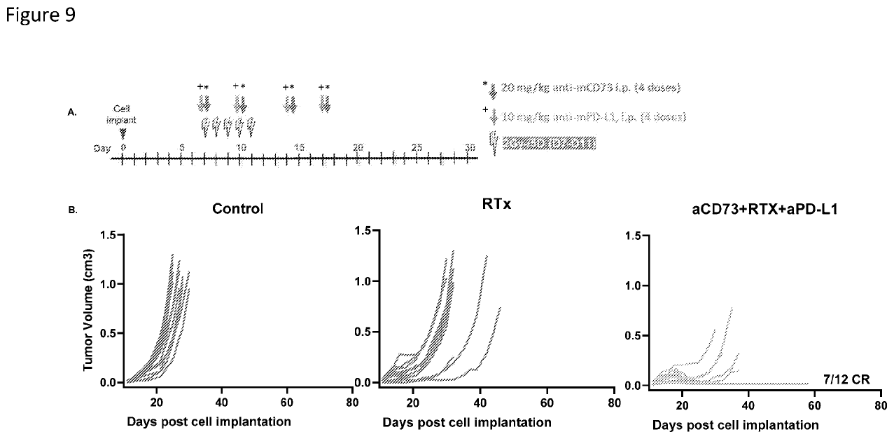

Previously published preclinical data highlights a role for CD73 within radiotherapy responsiveness in cancer (Wennerberg E, et al., Cancer Immunol Res 2020 8:465-78; Tsukui H, et al., BMC Cancer 2020; 20; and Nguyen A M, et al., Molecular & Cellular Proteomics 2020; 19:375-89). Whether addition of aCD73+aPD-L1 treatment would also augment radiotherapy responses in line with the chemotherapy data was explored. The MC38 model of colorectal cancer was used to test the effects that was already established for fractionated radiotherapy regimen, employing a concurrent approach in this experiment, i.e., all the treatments starting on the same day (schematic FIG. 9A). The tumors were enrolled once they were between 70-120 mm³ for the intervention treatment. The data confirmed a potent effect (p=0.0001), Kruskal-Wallis test, from RTx+aCD73+aPD-L1 treatment of MC38 tumor bearing mice as shown in FIG. 9B.

### Example 8

### Optimal Scheduling With aCD73, aPD-L1 in MC38 Syngeneic Mouse Model

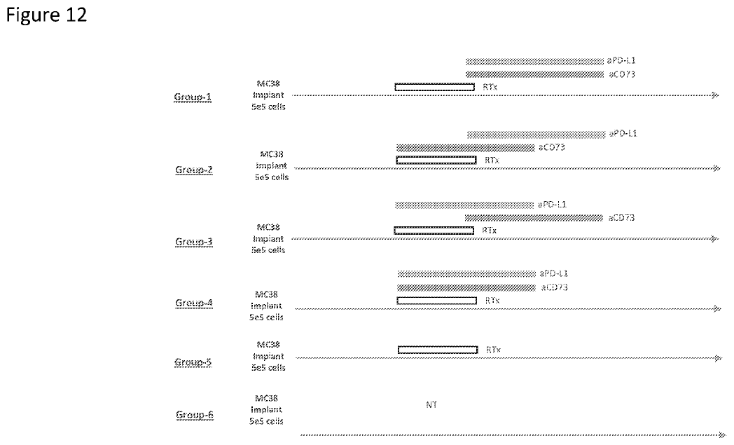

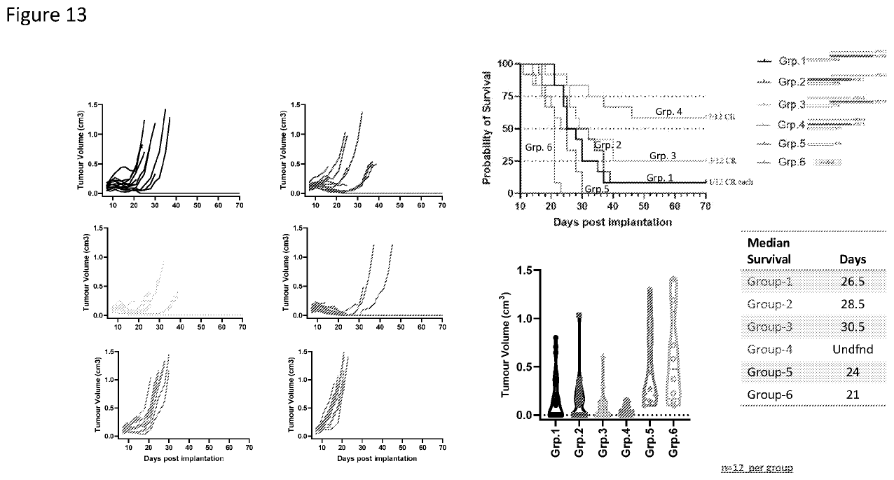

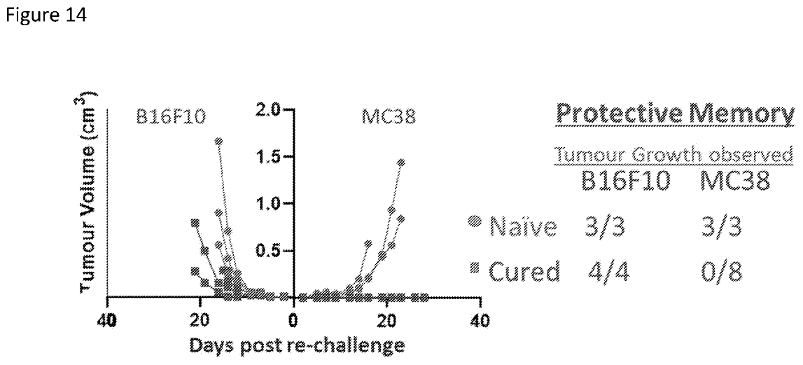

The effect of timing of aCD73, aPD-L1 and radiotherapy was also determined using a MC38 syngeneic mouse model. Six groups of mice were implanted with 5×10⁵ cells as shown in FIG. 12. As shown in FIG. 13, mice that were treated with aCD73, aPD-L1, and RTx concurrently showed the highest level of tumor inhibition and subsequent probability of survival. This also correlated with an induction of a protective memory response upon rechallenge using B16F10 and MC38 mouse models (FIG. 14).

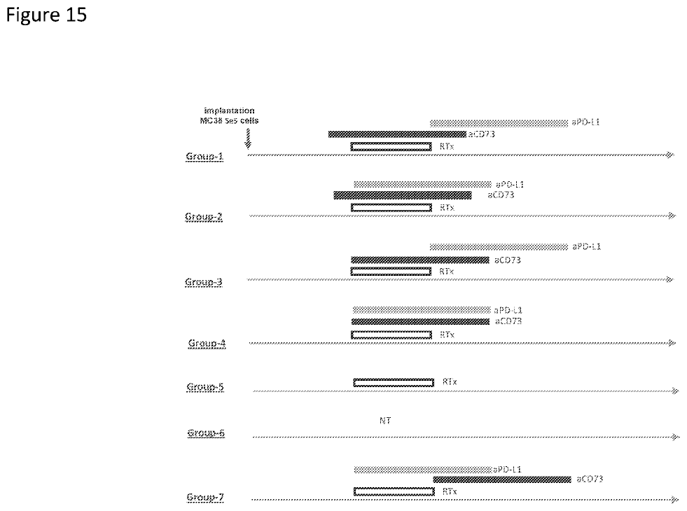

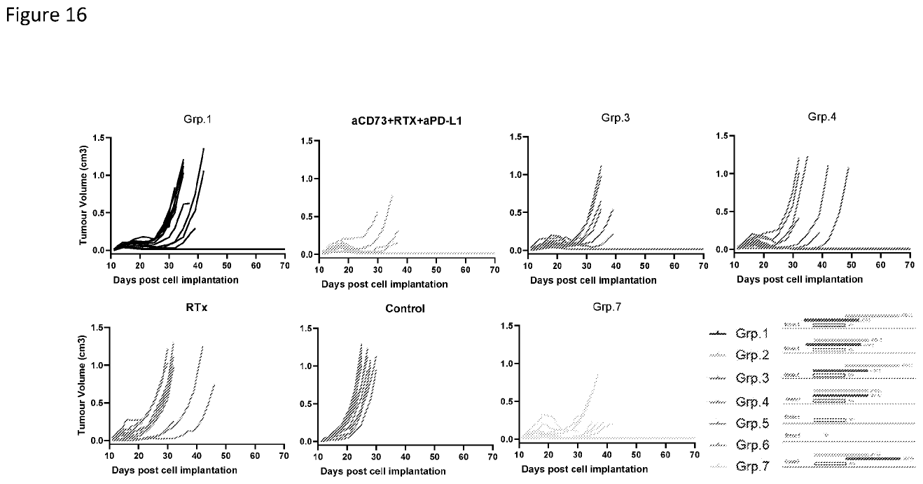

To determine whether there was an effect on tumor volume when the timing of the individual aCD73, aPD-L1, and radiotherapy were administered, MC38 cells were implanted as before and the timing of the individual therapies was mapped as shown in FIG. 15. Interestingly, administration of aCD73 therapy prior to aPD-L1 and RTx showed the greatest decrease in tumor volume, which correlated to the highest probability of survival (FIG. 16).

A growing body of evidence supports an immunosuppressive role for extracellular adenosine within the tumor microenvironment; with adenosine related gene signatures linked to poor outcome and reduced response to T-cell checkpoint inhibiting drugs in several indications (Sidders et al.,; and Allard D, et al., Immunol Lett 2019; 205:31-9). Molecules targeting extracellular nodes within the adenosine creation pathway have gained traction in the clinical development space, with Oleclumab now in Ph3 of clinical development in combination with Durvalumab in stage 3 NSCLC (Non-Small Cell Lung Cancer) patients previously treated with chemo-radiotherapy (A Global Study to Assess the Effects of Durvalumab With Oleclumab or Durvalumab With Monalizumab Following Concurrent Chemoradiation in Patients With Stage III Unresectable Non-Small Cell Lung Cancer-Full Text View-ClinicalTrials.gov).

Despite the widespread assumption that CD73 inhibition will combine beneficially with cytotoxic treatments that promote extracellular ATP release via cell death, there is a paucity of published preclinical data to support this. The data presented herein, exploring the effects of CD73 inhibition in concert with chemotherapy and PD-L1 inhibition, highlight additivity and novel biological effects mediated by inclusion of CD73 blockade. To this end, a combination comprising aCD73+aPD-L1+5FU+OHP afforded enhanced efficacy in two mouse models of cancer (colorectal and sarcoma). Combination therapy activity was contingent on CD8 T-cells, as judged by the effect of a CD8 depleting antibody in the MCA205 model. These data infer a critical contribution from the cell mediated arm of the murine immune system in the anti-tumor effect engendered. Consistent with this, RNAseq analysis confirmed aCD73+aPD-L1+5FU+OHP drove elevated tumoral abundances of cytotoxic lymphocytes and other key immune-cells, such as myeloid dendritic cells and B cells. Substituting OHP+5FU with a taxane backbone, i.e. DTX, was also explored and confirmed a similarly enhanced efficacy profile was attained in the CT26 model. These data support complementarity of Oleclumab and aPD-L1 antibodies, with both Platin and Taxane based chemotherapy backbones. The data also highlight additivity with radiotherapy in the MC38 model; extending the findings of Wennerberg et al. (Cancer Immunol Res 2020; 8:465-78) to colorectal models and augmenting the interventional strategies for inhibition of the PD-1/PD-L1 axis.

Mechanistically, aCD73 antibody rapidly lowered CD73 expression within CT26 tumors and modulated extracellular adenosine levels in manner consistent with its proposed mechanism of action. Imaging mass cytometry of the same samples did, however, pick up changes in CAF and TAM markers, that may reflect direct or downstream effects of aCD73 treatment. Such observations align with other publications linking CD73 inhibition to tumoral macrophage and fibroblast representation (Magagna I, et al., Cancers (Basel) 2021; 13.; Yu M, et al., Nat Commun 2020; 11).

Monotherapy 5FU+OHP treatment retarded tumor growth in a proportion of drug recipients bearing CT26/MCA205 tumors. A notable finding from the current studies was the breadth of immune-pathway gene modulation following 5FU+OHP treatment of CT26 tumors. These included Type 1 and 2 interferons, gene signatures associated with cytotoxic lymphocytes and effector molecules and their related receptors. Such observations align with many of the known immunomodulatory effects of these chemotherapies within in vitro model systems (Siew Y Y, et al., Int Immunol 2015; 27:621-32) and now extend the findings concerning effects in vivo (Dosset M, et al., Oncoimmunology 2018; 7). In particular, the data presented herein have identified 5FU+OHP drives type I interferon pathways, which are known to exert wide ranging effects on immune and cancer cells within the tumor microenvironment (Zitvogel L, et al., Nat Rev Immunol 2015; 15:405-14). Specific genes modulated by monotherapy 5FU+OHP treatment included IFN-Y, CCL3, CCL8, Lag-3 and granzyme B (upregulated) with downregulation of CXCL2, IL-1b, CD103 and XCR1. 5FU+OHP treatment elevated ARORA2 gene expression, although ARORA2 upregulation was counteracted when either aPD-L1 or aCD73 Mabs were combined with 5FU+OHP treatment. Gene signatures associated with proinflammation, STAT5 pathway activation and chemotaxis were only significantly upregulated once a CD73 blockade was applied with the OHP+5FU treatment (Table 2). GDF-15 is a pleotropic cytokine of emerging interest in cancer (Wischhusen J, et al., Front Immunol 2020; 11) and GDF15 transcript levels in CT26 tumors were markedly elevated by 5FU+OHP containing treatments; mirroring findings from other mouse models and human cancer patients on platinum therapy (Breen D M, et al., Cell Metab 2020; 32:938-950.e6).

In contrast to the tumor RNAseq data obtained from mice treated with either of the IO agents alone (e.g., as monotherapies), the combination of aCD73 and aPD-L1 resulted in 1236 differentially expressed genes; highlighting broader transcriptomic changes associated with antibody mediated targeting of multiple inhibitory checkpoints within the tumor microenvironment. The IO ‘doublet’ was noted for its activation of pathways and gene families linked to inflammation, myeloid leukocyte migration, cell chemotaxis, cytokine-cytokine receptor interactions (including TNF), chemokine signaling pathways and complement and coagulation cascades. The pathways and processes influenced by the IO combination (aCD73+aPD-L1) were mostly distinct from those influenced by 5FU+OHP treatment; with potential for complementarity if overlayed within a combinatorial therapeutic paradigm. Notably, the ‘doublet’ IO combination (aCD73+aPD-L1) afforded less efficient activation of type 1 interferon pathway genes; known to correlate with favorable disease outcomes in patients with many forms of cancer and mediate a range of beneficial immunomodulatory effects within the tumor microenvironment (Zitvogel L, et al., Nat Rev Immunol 2015; 15:405-14).

Despite the detected transcriptomic changes, it is clear that ‘purist’ small/large molecule approaches (5FU+OHP chemotherapy or IO combinations) failed to attain the same level of efficacy afforded by combined use of 5FU+OHP with aCD73 and aPD-L1. It was therefore informative to drill down into the specific differences in the transcriptomes that could account for this, as these genes and these signatures might be useful beyond the context of adenosine pathway modulation. Strikingly, addition of aCD73 to 5FU+OHP +aPD-L1 significantly upregulated CXCR3 in the CT26 tumor microenvironment. CXCR3 is the cognate receptor on activated T-cells for the IFN induced chemokines CXCL9-11; its upregulation being coincident with elevated abundances of T-cells and increased expression of interferon activated chemokines in the CT26 tumors of mice that received 5FU+OHP, or combinations containing that chemotherapy. Recent publications highlight the importance of tumoral chemotaxis of CXCR3 bearing T-cells for preclinical and clinical responsiveness to T-cell checkpoint inhibitors (Marcovecchio P M, et al., J Immunother Cancer 2021; 9; Qu Y, et al., Cell Rep 2020; 32; Chow M T, et al., Immunity 2019; 50:1498-1512.e5). Elevated expression of Pdcd1 (PD-1) infer tumoral T-cells in those CT26 tumor bearing mice are likely activated and/or exhausted and strongly support the inclusion of aPD-L1 to counteract adaptive immune-resistance to anti-tumor effects. Remarkably, the aPD-L1 and IO containing combinations markedly upregulated 15-lipoxygenases (15-LOXs), which have been implicated in various macrophage functions including efferocytosis and ferroptosis. This is interesting in that Snodgrass et al., (Front Immunol 2018; 9) identified a novel role for ALOX15 in CCL17 production in human macrophages; important to note in light of the obvious increase in CCL17 expression in the most protective therapeutic formats.

The regulation of genes linked to dendritic cell biology and antigen presentation (MHC II, Itgae, Itgax, DCstamp, TARMI, CD301) is particularly interesting and consistent with the work of others exploring CD73 inhibition in the context of radiotherapy4 and adenosine pathway blockade in general. Wennerberg et al. (Cancer Immunol Res. 2020) already highlighted the critical role of radiotherapy induced type 1 interferons for reprofiling the tumor microenvironment, particularly with respect to cDCI's. The same group highlighted the complementarity for CD73 blockade with radiotherapy, with a critical role for aCD73 treatment in tumors with suboptimal induction of radiotherapy elicited type 1 interferons. The data presented herein seem broadly in line with their findings. Consistent with this, genes for MHCII molecules and the macrophage galactose C-type lectin (MGL/CD301), expressed on DCs, were upregulated within the CT26 TME. CD301 is thought to participate in the recognition of molecules from both altered self and pathogens due to its monosaccharide specificity for Gal and N-acetylgalactosamine30. T cell-interacting, activating receptor on myeloid cells-1 (TARM1; gene symbol Tarm1) is a recently identified LILR family member encoded within the leukocyte receptor complex. TARM1 is expressed by and is required for the activation of DCs; Tarml was highly expressed in inflammatory-type (I-A/I-E+Ly6C+CD11b+CD11c+) DCs in draining LNs (dLNs) after induction of CIA in Tarm1+/−mice31. Another novel finding was the ability of the aCD73 containing combinations to uniquely drive high levels of Ear2 expression. Ear2 is an RNase and also forms part of a 14 gene signature expressed by non-classical monocytes (Ma R Y, et al., Trends Immunol 2022; 43:546-63). Emerging evidence points to an immunomodulatory role for extracellular RNase molecules, thereby acting as alarmins (Lu L, et al., Front Immunol 2018; 9:1012). Similarly, tumoral RNase2a expression was significantly increased by the combination therapy. An effect on NOX2 (gene Cybb) was also noted, and potentially noteworthy, considering its fundamental role in conferring macrophages with the ability to respond to extracellular ATP stimulation with robust changes in cellular oxidation (Moore S F, et al., Journal of Immunology 2009; 183:3302-8) and its role in modulating ATM kinase activation in macrophages and effectiveness to radiation therapy (Wu Q, et al., Cell Death Differ 2017; 24:1632-44). Macrophage lectin-like oxidized LDL receptor-1 (LOX-1/OLR1) was also upregulated in CT26 tumors from mice treated with 5FU+OHP+aCD73+aPD-L1. This receptor is known to sense heat-shock proteins and is markedly upregulated by TLR agonists and other proinflammatory stimuli.

The effect on B cell representation within the CT26 tumor is also comment worthy (FIG. 7A and 7C). B-cell related genes, for example JChain, CXCR5 and CXCL 13 were clearly modulated when aCD73 was included in the 5FU+OHP+aPD-L1 combination (FIG. 11). Although the influence of B-cell biology is less well understood than CD8s in cancer, there is growing interest in the role of B cells in human tumor microenvironments (Fridman W H, et al., Journal of Experimental Medicine 2021; 218.; Griss J, et al., Nature Communications 2019; 10:1-14.; Bruni D, et al., Nature Reviews Cancer 2020; 20:662-80). The murine B cell related data is also concordant with recent publications concerning direct effects, of CD73 inhibiting antibodies, on human B-cells (Hair J, et al., Cancer Res 2021; 81:1695-1695.; Luke J, et al., J Immunother Cancer 2021; 9: A729-A729). These mechanistic data align nicely to the enhanced survival rates and tumor growth control profiles in animals dosed with 5FU+OHP+aCD73+aPD-L1.

What is claimed is:

1. A method of inhibiting tumor growth in a subject in need thereof comprising administering to the subject a therapeutically effective amount of a PD-L1 inhibitor in combination with chemotherapy and/or radiotherapy; wherein the subject has decreased CD73 protein or CD73 activity levels compared to a normal subject.

2. A method of treating cancer in a subject in need thereof comprising administering to the subject a therapeutically effective amount of a PD-L1 inhibitor in combination with chemotherapy and/or radiotherapy; wherein the subject has decreased CD73 protein or CD73 activity levels compared to a normal subject.

3. A method of producing a protective tumor memory response in a subject comprising administering to the subject a therapeutically effective amount of a PD-L1 inhibitor in combination with chemotherapy and/or radiotherapy; wherein the subject has decreased CD73 protein or CD73 activity levels compared to a normal subject.

4. A method of inhibiting tumor growth in a subject in need thereof comprising administering to the subject a therapeutically effective amount of a CD73 inhibitor, a PD-L1 inhibitor, and chemotherapy and/or radiotherapy.

5. A method of treating cancer in a subject in need thereof comprising administering to the subject a therapeutically effective amount of a CD73 inhibitor, a PD-L1 inhibitor, and chemotherapy and/or radiotherapy.

6. A method of producing a protective tumor memory response in a subject comprising administering to the subject a therapeutically effective amount of a CD73 inhibitor, a PD-L1 inhibitor, and chemotherapy and/or radiotherapy.

7. The method of any one of claims 1-6, wherein the PD-L1 inhibitor, and the chemotherapy and/or radiotherapy are administered concurrently.

8. The method of any one of claims 1-6, wherein the PD-L1 inhibitor, and the chemotherapy and/or radiotherapy are administered sequentially.

9. The method of any one of claims 4-6, wherein the CD73 inhibitor is administered prior to administration of the PD-L1 inhibitor, and the chemotherapy and/or radiotherapy.

10. The method of any one of claims 1-9, wherein the chemotherapy is docetaxel, 5-fluorouracil, and/or oxaliplatin.

11. The method of any one of claims 1-10, wherein the PD-L1 inhibitor is an anti-PD-L1 antibody or an antigen-binding fragment thereof.

12. The method of claim 11, wherein the anti-PD-L1 antibody or antigen-binding fragment thereof comprising: (a) a heavy chain (HC) CDR1 comprising the amino acid sequence of SEQ ID NO:1, a HC CDR2 comprising the amino acid sequence of SEQ ID NO:2, and a HC CDR3 comprising the amino acid sequence of SEQ ID NO:3; and a light chain (LC) CDR1 comprising the amino acid sequence of SEQ ID NO:4, a LC CDR2 comprising the amino acid sequence of SEQ ID NO:5, and a LC CDR3 comprising the amino acid sequence of SEQ ID NO:6.

13. The method of claim 11 or 12, wherein the anti-PD-L1 antibody or antigen binding fragment thereof comprises a HC variable domain (VH) comprising the amino acid sequence of SEQ ID NO:7, and a LC variable domain (VL) comprising the amino acid sequence of SEQ ID NO:8.

14. The method of any of claims 11-13, wherein the anti-PD-L1 antibody is durvalumab.

15. The method of any one of claims 4-14, wherein the CD73 inhibitor is an anti-CD73 antibody or antigen-binding fragment thereof.

16. The method of claim 15, wherein the anti-CD73 antibody or antigen-binding fragment thereof comprising: (a) a HC CDR1 comprising the amino acid sequence of SEQ ID NO: 9, a HC CDR2 comprising the amino acid sequence of SEQ ID NO: 10, and a HC CDR3 comprising the amino acid sequence of SEQ ID NO:11; and a LC CDR1 comprising the amino acid sequence of SEQ ID NO: 12, a LC CDR2 comprising the amino acid sequence of SEQ ID NO:13, and a LC CDR3 comprising the amino acid sequence of SEQ ID NO: 14.

17. The method of claim 15 or 16, wherein the anti-CD73 antibody or antigen binding fragment thereof comprises a HC variable domain (VH) comprising the amino acid sequence of SEQ ID NO:15, and a LC variable domain (VL) comprising the amino acid sequence of SEQ ID NO:16.

18. The method of any one of claims 15-17, wherein the anti-CD73 antibody or antigen binding fragment thereof comprises a HC comprising the amino acid sequence of SEQ ID NO: 17, and a LC comprising the amino acid sequence of SEQ ID NO:18.

19. The method of any of claims 11-13, wherein the anti-CD73 antibody is oleclumab.

20. The method of any one of claims 1-19, wherein administration results in upregulation of CXCR3 in the tumor microenvironment.

21. The method of any one of claims 1-3 and 7-20, wherein CD73 protein or CD73 activity levels are determined by immunohistochemistry (IHC), imaging mass cytometry (IMC), or mass spectroscopy imaging (MSI).

22. The method of any one of claims 1-21, wherein the tumor or cancer is a solid tumor or a cancer resulting from a solid tumor growth.

23. The method of claim 22, wherein the solid tumor is a lung tumor, breast tumor, colon tumor, bladder tumor, prostate tumor, colorectal tumor, head and neck tumor, liver tumor, or a pancreatic tumor.

24. The method of claim 23, wherein the lung tumor is a non-small cell lung tumor.

25. The method of any one of claims 1-24, wherein the subject is a human.

26. Use of a CD73 inhibitor, a PD-L1 inhibitor, and chemotherapy and/or radiotherapy of any one of claims 1-25 for treating cancer in a subject in need thereof.


SEQUENCE LISTING (18 sequences)

SEQ ID NO: 1 - protein, 10 aa, synthetic construct
  source 1..10: note = Heavy Chain CDR1 Amino Acid Sequence; Anti-PD-L1 Antibody Durvalumab
GFTFSRYWMS

SEQ ID NO: 2 - protein, 17 aa, synthetic construct
  source 1..17: note = Heavy Chain CDR2 Amino Acid Sequence; Anti-PD-L1 Antibody Durvalumab
NIKQDGSEKYYVDSVKG

SEQ ID NO: 3 - protein, 12 aa, synthetic construct
  source 1..12: note = Heavy Chain CDR3 Amino Acid Sequence; Anti-PD-L1 Antibody Durvalumab
EGGWFGELAFDY

SEQ ID NO: 4 - protein, 12 aa, synthetic construct
  source 1..12: note = Light Chain CDR1 Amino Acid Sequence; Anti-PD-L1 Antibody Durvalumab
RASQRVSSSYLA

SEQ ID NO: 5 - protein, 7 aa, synthetic construct
  source 1..7: note = Light Chain CDR2 Amino Acid Sequence; Anti-PD-L1 Antibody Durvalumab
DASSRAT

SEQ ID NO: 6 - protein, 9 aa, synthetic construct
  source 1..9: note = Light Chain CDR3 Amino Acid Sequence; Anti-PD-L1 Antibody Durvalumab
QQYGSLPWT

SEQ ID NO: 7 - protein, 121 aa, synthetic construct
  source 1..121: note = Heavy Chain Variable Domain (VH) Amino Acid Sequence; Anti-PD-L1 Antibody Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 8 - protein, 108 aa, synthetic construct
  source 1..108: note = Light Chain Variable Domain (VL) Amino Acid Sequence; Anti-PD-L1 Antibody Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 9 - protein, 5 aa, synthetic construct
  source 1..5: note = Heavy Chain CDR1 Amino Acid Sequence; Anti-CD73 Antibody Olecumab
SYAYS

SEQ ID NO: 10 - protein, 17 aa, synthetic construct
  source 1..17: note = Heavy Chain CDR2 Amino Acid Sequence; Anti-CD73 Antibody Olecumab
AISGSGGRTYYADSVKG

SEQ ID NO: 11 - protein, 8 aa, synthetic construct
  source 1..8: note = Heavy Chain CDR3 Amino Acid Sequence; Anti-CD73 Antibody Olecumab
LGYGRVDE

SEQ ID NO: 12 - protein, 13 aa, synthetic construct
  source 1..13: note = Light Chain CDR1 Amino Acid Sequence; Anti-CD73 Antibody Olecumab
SGSLSNIGRNPVN

SEQ ID NO: 13 - protein, 7 aa, synthetic construct
  source 1..7: note = Light Chain CDR2 Amino Acid Sequence; Anti-CD73 Antibody Olecumab
LDNLRLS

SEQ ID NO: 14 - protein, 11 aa, synthetic construct
  source 1..11: note = Light Chain CDR3 Amino Acid Sequence; Anti-CD73 Antibody Olecumab
ATWDDSHPGWT

SEQ ID NO: 15 - protein, 117 aa, synthetic construct
  source 1..117: note = Heavy Chain Variable Domain (VH) Amino Acid Sequence; Anti-CD73 Antibody Olecumab
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 16 - protein, 110 aa, synthetic construct
  source 1..110: note = Light Chain Variable Domain (VL) Amino Acid Sequence; Anti-CD73 Antibody Olecumab
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 17 - protein, 447 aa, synthetic construct
  source 1..447: note = Heavy Chain Amino Acid Sequence; Anti-CD73 Antibody Olecumab
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 216 aa, synthetic construct
  source 1..216: note = Light Chain Amino Acid Sequence; Anti-CD73 Antibody Olecumab
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS